(19) United States
(12) Patent Application Publication
(10) Pub. No.: US 2014/0187499 A1
(43) Pub. Date: Jul. 3, 2014

(54) SUBSTITUTED ANIONIC COMPOUNDS CONSISTING OF A BACKBONE MADE UP OF A DISCRETE NUMBER OF SACCHARIDE UNITS

(71) Applicant: ADOCIA, Lyon (FR)

(72) Inventors: Gérard SOULA, Meyzieu (FR); Emmanuel DAUTY, Lyon (FR); Richard CHARVET, Rillieux-la-Pape (FR)

(21) Appl. No.: 14/079,437

(22) Filed: Nov. 13, 2013

Related U.S. Application Data
(60) Provisional application No. 61/726,349, filed on Nov. 14, 2012.
(60) Provisional application No. 61/725,775, filed on Nov. 13, 2012.
(60) Provisional application No. 61/763,766, filed on Feb. 12, 2013.

(30) Foreign Application Priority Data
Nov. 13, 2012 (FR) 12/60808
Nov. 14, 2012 (FR) 12/60855
Feb. 12, 2013 (FR) 13/51199

(51) Int. Cl. A61K 47/26; A61K 47/18

(52) U.S. Cl. CPC A61K 47/26; A61K 47/183
USPC 514/20.9; 536/17.9; 514/777; 536/17.4; 536/5; 536/55.1; 514/773; 564/153; 514/1.1

(57) ABSTRACT

The invention relates to substituted anionic compounds consisting of a backbone made up of a discrete number u of between 1 and 8 (1≦u≦8) of identical or different saccharide units, linked via identical or different glycosidic bonds, said saccharide units being chosen from the group consisting of pentoses, hexoses, uronic acids, N-acetylhexosamines in cyclic form or in open reduced form, which are randomly substituted. It also relates to the process for the preparation thereof and to the pharmaceutical compositions comprising same.

The present invention relates to anionic compounds intended for therapeutic and/or prophylactic use, for the administration of an active ingredient or active ingredients to humans or to animals.

The anionic compounds according to the invention of which the backbone consists of saccharide units comprising carboxyl groups are, owing to their structure and their biocompatibility, undoubtedly of interest for the pharmaceutical industry, in particular for stabilizing active ingredients, for example proteins.

Polysaccharides and/or oligosaccharides which have properties of creating interactions with active ingredients, for example proteins, are known from WO 2008/038111 and WO 2010/041119, which are patent applications filed in the name of Adocia.

In these patent applications, the polymers or oligomers are defined in terms of their degree of polymerization DP, which is the average number of repeating units (monomers) per polymer chain. It is calculated by dividing the number-average molar mass by the average mass of the repeated unit. They are also defined in terms of the chain length distribution, also called the polydispersity index (Ip).

These polymers are therefore compounds consisting of chains of which the lengths are statistically variable, which are highly rich in possible sites of interaction with protein active ingredients. This multiple-interaction potential could create a lack of specificity in terms of interaction, whereas a smaller, better defined molecule could make it possible to be more specific in this respect.

Moreover, a polymer chain can interact with various sites present on a protein ingredient, but can also, owing to the chain length, interact with several protein ingredients, thereby leading to a bridging phenomenon. This bridging phenomenon may, for example, result in aggregation of the proteins or in an increase in viscosity. The use of a small molecule with a well-defined backbone makes it possible to minimize these bridging phenomena.

In addition, a molecule with a well-defined backbone is generally more readily traceable (MS/MS, for example) in biological media during pharmacokinetic or ADME (administration, distribution, metabolism, elimination) experiments compared with a polymer which generally gives a very diffuse signal with a high background noise in mass spectrometry.

In contrast, it is not out of the question for a well-defined and shorter molecule to possibly exhibit a shortage of possible sites of interaction with protein active ingredients.

Notwithstanding their perfectly defined structure, the anionic compounds according to the invention consisting of a backbone made up of a discrete number u of between 1 and 8 (1≦u≦8) of identical or different saccharide units also have the property of creating interactions with active ingredients, protein active ingredients for example.

They nevertheless have particular properties with respect to certain active ingredients which make them candidates of choice for preparing pharmaceutical formulations.

The functionalization of these anionic compounds with carboxyl groups advantageously makes it possible to modulate the interaction forces involved between the anionic compound and the active ingredient.

By virtue of the defined structure of the backbone, the functionalization is easier and more precise and the nature of the anionic compounds obtained is therefore more homogeneous than when the backbone is of polymeric nature.

The present invention thus aims to provide anionic compounds intended for the stabilization, administration and delivery of active ingredients, which can be prepared by methods that are relatively simple to carry out. The objective of the present invention is thus to provide anionic compounds capable of enabling the stabilization, administration and delivery of a large diversity of active ingredients.

The invention is also directed toward the obtaining of anionic compounds which can exhibit biodegradability that is sufficiently rapid and suitable for their use in the preparation of a broad category of pharmaceutical formulations, including for medicaments intended for chronic and/or high-frequency administration. In addition to the requirement of biodegradability that can be modulated after administration, the invention aims to provide anionic compounds which comply with the constrains established by the pharmaceutical industry, in particular in terms of stability under normal preservation and storage conditions, and in particular in solution.

As will be demonstrated in the examples, the substituted anionic compounds according to the invention make it possible to prepare solutions which are nonturbid in the presence of certain “model” proteins for formulation, such as lysozyme, which is not possible with certain polymeric compounds, but are nevertheless capable of interacting with model proteins such as albumin. This duality makes it possible to modulate their properties and to obtain good excipient candidates for the formulation of protein active ingredients without the drawbacks exhibited by some of the compounds described in the prior art.

The present invention relates to substituted anionic compounds, in isolated form or as a mixture, consisting of a backbone made up of a discrete number u of between 1 and 8 (1≦u≦8) of identical or different saccharide units, linked via identical or different glycosidic bonds, said saccharide units being chosen from the group consisting of pentoses, hexoses, uronic acids, N-acetylhexosamines in cyclic form or in open reduced form, characterized in that they are substituted with:

a) at least one substituent of general formula I:

—[R₁]ₐ-[[Q]-[R₂]ₙ]ₘ formula I
the substituents being identical or different when there are at least two substituents, in which:
if n is equal to 0, then the radical -[Q]- is derived from a C₃ to C₁₅ carbon-based chain which is optionally branched or substituted, optionally unsaturated and/or optionally comprising one or more ring(s) and/or comprising at least one heteroatom chosen from O, N and S and at least one function L chosen from amine and alcohol functions, said radical -[Q]- being attached to the backbone of the compound by means of a linker arm R₁ to which it is bonded via a function T, or directly bonded to the backbone via a function G,
if n is equal to 1 or 2, then the radical -[Q]- is derived from a C₂ to C₁₅ carbon-based chain which is optionally branched or substituted, optionally unsaturated and/or optionally comprising one or more ring(s) and/or comprising at least one heteroatom chosen from O, N and S and at least one function L chosen from amine and alcohol functions and bearing n radical(s) R₂, said radical -[Q]-being attached to the backbone of the compound by means of a linker arm R₁ to which it is bonded via a function T, or directly bonded to the backbone via a function G,
the radical —R₁— being:
   either a bond and then a=0, and the radical -[Q]- is directly bonded to the backbone via a function G,
   or a C₂ to C₁₅ carbon-based chain, and then a=1, which is optionally substituted and/or comprising at least one heteroatom chosen from O, N and S and at least one acid function before the reaction with the radical -[Q]-, said chain being bonded to the radical -[Q]- via a function T resulting from the reaction of the acid function of the radical —R₁— with an alcohol or amine function of the precursor of the radical -[Q]-, and said radical R₁ is attached to the backbone by means of a function F resulting from a reaction between a hydroxyl function or a carboxylic acid function borne by the backbone and a function or a substituent borne by the precursor of the radical —R₁—,
the radical —R₂ is a C₁ to C₃₀ carbon-based chain which is optionally branched or substituted, optionally unsaturated and/or optionally comprising one or more ring(s) and/or one or more heteroatom(s) chosen from O, N and S; it forms, with the radical -[Q]-, a function Z resulting from a reaction between the alcohol, amine or acid functions borne by the precursors of the radical —R₂ and of the radical -[Q]-.
F is a function chosen from ether, ester, amide or carbamate functions,
T is a function chosen from amide or ester functions,
Z is a function chosen from ester, carbamate, amide or ether functions,
G is a function chosen from ester, amide or carbamate functions,
n is equal to 0, 1 or 2,
m is equal to 1 or 2,
the degree of substitution of the saccharide units, j, with —[R₁]ₐ-[[AA]-[R₂]ₙ]ₘ being between 0.01 and 6, 0.01≦j≦6;

b) and, optionally, one or more substituents —R′₁,

the substituent —R′₁ being a C₂ to C₁₅ carbon-based chain which is optionally substituted and/or comprising at least one heteroatom chosen from O, N and S and at least one acid function in the form of an alkali metal cation salt, said chain being bonded to the backbone via a function F′ resulting from a reaction between a hydroxyl function or a carboxylic acid function borne by the backbone and a function or a substituent borne by the precursor of the substituent —R′₁,
the degree of substitution of the saccharide units, i, with being between 0 and 6-j, 0≦i≦6-j and,
if n≠0 and if the backbone does not bear anionic charges before substitution, then i≠0,
—R′₁ identical to or different than —R₁—,
the free salifiable acid functions borne by —R′₁— are in the form of alkali metal cation salts,
F′ is a function chosen from ether, ester, amide or carbamate functions,
F, F′, T, Z and G being identical or different,
i+j≦6.

In one embodiment, u is between 3 and 8.

In one embodiment, u is between 3 and 5.

In one embodiment, u is equal to 3.

In one embodiment, L is an amine function.

In one embodiment, L is an alcohol function.

In one embodiment, 0.05≦j≦6.

In one embodiment, 0.05≦j≦4.

In one embodiment, 0.1≦j≦3.

In one embodiment, 0.1≦j≦2.

In one embodiment, 0.2≦j≦1.5.

In one embodiment, 0.3≦j≦1.2.

In one embodiment, 0.5≦j≦1.2.

In one embodiment, 0.6≦j≦1.1.

In one embodiment, 0.25≦i≦3.

In one embodiment, 0.5≦i≦2.5.

In one embodiment, 0.6≦i≦2.

In one embodiment, 0.6≦i≦1.5.

In one embodiment, 0.6≦i≦1.1.

In one embodiment, 0.3≦i+j≦6.

In one embodiment, 0.5≦i+j≦4.

In one embodiment, 0.5≦i+j≦3.

In one embodiment, 0.5≦i+j≦2.5.

In one embodiment, 1≦i+j≦2.

In one embodiment, m=2.

In one embodiment, m=1.

In one embodiment, n=2.

In one embodiment, n=1.

In one embodiment, n=0.

In one embodiment, the anionic compounds according to the invention are characterized in that the radical -[Q]- is derived from an alpha-amino acid.

In one embodiment, the anionic compounds according to the invention are characterized in that the radical -[Q]- is derived from an alpha-amino acid and n=0.

In one embodiment, the anionic compounds according to the invention are characterized in that the alpha-amino acid is chosen from the group comprising alpha-methylphenylalanine, alpha-methyltyrosine, O-methyltyrosine, alpha-phenylglycine, 4-hydroxyphenylglycine and 3,5-dihydroxyphenylglycine, in their L, D or racemic forms.

In one embodiment, the anionic compounds according to the invention are characterized in that the alpha-amino acid is chosen from natural alpha-amino acids.

In one embodiment, the anionic compounds according to the invention are characterized in that the natural alpha-amino acid is chosen from hydrophobic amino acids chosen from the group comprising tryptophan, leucine, alanine, isoleucine, glycine, phenylalanine, tyrosine and valine, in their L, D or racemic forms.

In one embodiment, the anionic compounds according to the invention are characterized in that the natural alpha-amino acid is chosen from polar amino acids chosen from the group comprising aspartic acid, glutamic acid, lysine, serine and threonine, in their L, D or racemic forms.

In one embodiment, the precursor of the radical -[Q]- is chosen from diamines.

In one embodiment, the precursor of the radical -[Q]- is chosen from diamines and n=1 or n=2.

In one embodiment, the diamines are chosen from the group consisting of ethylenediamine and lysine and its derivatives.

In one embodiment, the diamines are chosen from the group consisting of diethylene glycol diamine and triethylene glycol diamine.

In one embodiment, the precursor of the radical -[Q]- is chosen from amino alcohols.

In one embodiment, the precursor of the radical -[Q]- is chosen from amino alcohols and n=1 or n=2.

In one embodiment, the amino alcohols are chosen from the group consisting of ethanolamine, 2-aminopropanol, isopropanolamine, 3-amino-1,2-propanediol, diethanolamine, diisopropanolamine, tromethamine (Tris) and 2-(2-aminoethoxy)ethanol.

In one embodiment, the precursor of the radical -[Q]- is chosen from dialcohols.

In one embodiment, the precursor of the radical -[Q]- is chosen from dialcohols and n=1 or n=2.

In one embodiment, the dialcohols are chosen from the group consisting of glycerol, diglycerol and triglycerol.

In one embodiment, the dialcohol is triethanolamine.

In one embodiment, the dialcohols are chosen from the group consisting of diethylene glycol and triethylene glycol.

In one embodiment, the dialcohols are chosen from the group consisting of polyethylene glycols.

In one embodiment, the precursor of the radical -[Q]- is chosen from trialcohols.

In one embodiment, the trialcohol is triethanolamine.

In one embodiment, when the radical -[Q]- is chosen from amino acids, the present invention relates to substituted anionic compounds, in isolated form or as a mixture, consisting of a backbone made up of a discrete number u of between 1 and 8 (1≦u≦8) of identical or different saccharide units, linked via identical or different glycosidic bonds, said saccharide units being chosen from the group consisting of pentoses, hexoses, uronic acids, N-acetylhexosamines in cyclic form or in open reduced form, characterized in that they are substituted with:

a) at least one substituent of general formula II:

—[R₁]ₐ-[[AA]-[R₂]ₙ]ₘ formula II
the substituents being identical or different when there are at least two substituents, in which:
if n is equal to 0, then the radical -[AA]- denotes an amino acid residue comprising a C₃ to C₁₅ carbon-based chain directly bonded to the backbone via a function G′,
if n is equal to 1 or 2, then the radical -[AA]- denotes an amino acid residue comprising a C₂ to C₁₅ carbon-based chain bearing n radical(s) —R₂ attached to the backbone of the compound by means of a linker arm R₁ to which it is bonded via an amide function, or directly bonded to the backbone via a function G′,
the radical —R₁— being:
either a bond and then a=0, and the amino acid residue -[AA]- is directly bonded to the backbone via a function G′,
or a C₂ to C₁₅ carbon-based chain, and then a=1, which is optionally substituted and/or comprising at least one heteroatom chosen from O, N and S and at least one acid function before the reaction with the amino acid, said chain forming, with the amino acid residue -[AA]-, an amide function, and is attached to the backbone by means of a function F resulting from a reaction between a hydroxyl function or a carboxylic acid function borne by the backbone and a function or a substituent borne by the precursor of the radical —R₁—,
the radical —R₂ is a C₁ to C₃₀ carbon-based chain which is optionally branched or substituted, optionally unsaturated and/or optionally comprising one or more ring(s) and/or one or more heteroatom(s) chosen from O, N or S; it forms, with the amino acid residue -[AA]-, a function Z′ resulting from a reaction between a hydroxyl, acid or amine function borne by the precursor of the radical —R₂ and an acid, alcohol or amine function borne by the precursor of the radical -[AA]-,
F is a function chosen from ether, ester, amide or carbamate functions,
G′ is a function chosen from ester, amide or carbamate functions,
Z′ is a function chosen from ester, amide or carbamate functions,
n is equal to 0, 1 or 2,
m is equal to 1 or 2,
the degree of substitution of the saccharide units, j, with —[R₁]ₐ-[[AA]-[R₂]ₙ]ₘ being between 0.01 and 6, 0.01≦j≦6;

b) and, optionally, one or more substituents —R′₁,
the substituent —R′₁ being a C₂ to C₁₅ carbon-based chain which is optionally substituted and/or comprising at least one heteroatom chosen from O, N and S and at least one acid function in the form of an alkali metal cation salt, said chain being bonded to the backbone via a function F′ resulting from a reaction between a hydroxyl function or a carboxylic acid function borne by the backbone and a function or a substituent borne by the precursor of the substituent —R′₁,
the degree of substitution of the saccharide units, i, with —R′₁, being between 0 and 6-j, 0≦i≦6-j, and
   if n≠0 and if the backbone does not bear anionic charges before substitution, then i≠0,
—R′₁ identical to or different than —R₁—,
   the free salifiable acid functions borne by the substituent —R′₁ are in the form of alkali metal cation salts,
F′ is an ether, ester, amide or carbamate function,
F, F′, G′ and Z′ are identical or different,
i+j≦6.

In one embodiment, u is between 3 and 8.

In one embodiment, u is between 3 and 5.

In one embodiment, u is equal to 3.

In one embodiment, 0.05≦j≦6.

In one embodiment, 0.05≦j≦4.

In one embodiment, 0.1≦j≦3.

In one embodiment, 0.1≦j≦2.

In one embodiment, 0.2≦j≦1.5.

In one embodiment, 0.3≦j≦1.2.

In one embodiment, 0.5≦j≦1.2.

In one embodiment, 0.6≦j≦1.1.

In one embodiment, 0.25≦i≦3.

In one embodiment, 0.5≦i≦2.5.

In one embodiment, 0.6≦i≦2.

In one embodiment, 0.6≦i≦1.5.

In one embodiment, 0.6≦i≦1.1.

In one embodiment, 0.3≦i+j≦6.

In one embodiment, 0.5≦i+j≦4.

In one embodiment, 0.5≦i+j≦3.

In one embodiment, 0.5≦i+j≦2.5.

In one embodiment, 1≦i+j≦2.

In one embodiment, m=2.

In one embodiment, m=1.

In one embodiment, n=2.

In one embodiment, n=1.

In one embodiment, n=0.

In one embodiment, the present invention relates to substituted anionic compounds consisting of a backbone made up of a discrete number u of between 1 and 8 (1≦u≦8) of identical or different saccharide units, linked via identical or different glycosidic bonds, said saccharide units being chosen from the group consisting of pentoses, hexoses, uronic acids, N-acetylhaxoamines in cyclic form or in open reduced form, characterized in that they are randomly substituted with:

a) at least one substituent of general formula II:

—[R₁]ₐ-[[AA]-[R₂]ₙ]ₘ formula II
the substituents being identical or different when there are at least two substituents, in which:
the radical -[AA]- denotes an amino acid residue optionally bearing n radical(s) R₂ attached to the backbone of the compound by means of a linker arm R₁, or directly bonded to the backbone via a function G′,
—R₁— being:
   either a bond and then a=0,
   or a C₂ to C₁₅ carbon-based chain, and then a=1, which is optionally substituted and/or comprising at least one heteroatom chosen from O, N and S and at least one acid function before the reaction with the amino acid, said chain forming, with the amino acid residue -[AA]-, an amide bond, and is attached to the backbone by means of a function F resulting from a reaction between a hydroxyl function or a carboxylic acid function borne by the backbone and a function borne by the precursor of —R₁—,
the radical —R₂ is a C₁ to C₃₀ carbon-based chain which is optionally branched or substituted, optionally unsaturated and/or optionally comprising one or more ring(s) and/or one or more heteroatom(s) chosen from O, N and S; it forms, with the amino acid residue -[AA]-, a bond of ester, carbamate, amide or ether type resulting from a reaction between a function borne by —R₂ and a function borne by the precursor of the radical -[AA]-,
F is an ether, ester, amide or carbamate function,
G′ is an ester, amide or carbamate function,
n is equal to 0, 1 or 2,
m is equal to 1 or 2,
the degree of substitution, j, with —[R₁]ₐ-[[AA]-[R₂]ₙ]ₘ being between 0.01 and 6, 0.01≦j≦6;

b) and, optionally, one or more substituents —R′₁,
—R′₁ being a C₂ to C₁₅ carbon-based chain which is optionally substituted and/or comprising at least one heteroatom chosen from O, N and S and at least one acid function in the form of an alkali metal cation salt, said chain being bonded to the backbone via a function F′ resulting from a reaction between a hydroxyl function or a carboxylic acid function borne by the backbone and a function borne by the precursor of —R′₁,
the degree of substitution i, with —R′₁, being between 0 and 6-j, 0≦i≦6-j and,
   if n≠0 and if the backbone does not bear any anionic charges before substitution, then i≠0,
—R′₁ identical to or different than —R₁—,
   the free salifiable acid functions borne by R′₁ are in the form of alkali metal cation salts,
F′ is an ether, ester, amide or carbamate function,
F and F′ are identical or different,
i+j≦6.

In one embodiment, u is between 3 and 5.

In one embodiment, u is equal to 3.

In one embodiment, 0.05≦j≦6.

In one embodiment, 0.05≦j≦4.

In one embodiment, 0.1≦j≦3.

In one embodiment, 0.1≦j≦2.

In one embodiment, 0.2≦j≦1.5.

In one embodiment, 0.3≦j≦1.2.

In one embodiment, 0.5≦j≦1.2.

In one embodiment, 0.6≦j≦1.1.

In one embodiment, 0.25≦i≦3.

In one embodiment, 0.5≦i≦2.5.

In one embodiment, 0.6≦i≦2.

In one embodiment, 0.6≦i≦1.5.

In one embodiment, 0.6≦i≦1.1.

In one embodiment, 0.3≦i+j≦6.

In one embodiment, 0.5≦i+j≦4.

In one embodiment, 0.5≦i+j≦3.

In one embodiment, 0.5≦i+j≦2.5.

In one embodiment, 1≦i+j≦2.

In one embodiment, m=2.

In one embodiment, m=1.

In one embodiment, n=2.

In one embodiment, n=1.

In one embodiment, n=0.

In one embodiment, the substituted anionic compound is chosen from the substituted anionic compounds, in isolated form or as a mixture, consisting of a backbone made up of a discrete number u of between 1 and 8 (1≦u≦8) of identical or different saccharide units, linked via identical or different glycosidic bonds, said saccharide units being chosen from the group consisting of hexoses, in cyclic form or in open reduced form, characterized in that they are substituted with:

a) at least one substituent of general formula V:

—[R₁]ₐ-[AA]ₘ formula V
the substituents being identical or different when there are at least two substituents, in which:
the radical -[AA]- denotes an amino acid residue,
the radical —R₁— being:
   either a bond and then a=0, and the amino acid residue -[AA] is directly bonded to the backbone via a function Gₐ,
   or a C₂ or C₁₅ carbon-based chain, and then a=1, which is optionally substituted and/or comprising at least one heteroatom chosen from O, N and S and at least one acid function before the reaction with the amino acid, said chain forming, with the amino acid residue -[AA], an amide function, and is attached to the backbone by means of a function Fₐ resulting from a reaction between a hydroxyl function borne by the backbone and a function or a substituent borne by the precursor of the radical —R₁—,
Fₐ is a function chosen from ether, ester or carbamate functions,
Gₐ is a carbamate function,
m is equal to 1 or 2,
the degree of substitution of the saccharide units, j, with —[R₁]ₐ-[AA]ₘ being strictly greater than 0 and less than or equal to 6, 0<j≦6;

b) and, optionally, one or more substituents
the substituent —R′₁ being a C₂ to C₁₅ carbon-based chain which is optionally substituted and/or comprising at least one heteroatom chosen from O, N and S and at least one acid function in the form of an alkali metal cation salt, said chain being bonded to the backbone via a function F′ₐ resulting from a reaction between a hydroxyl function or a carboxylic acid function borne by the backbone and a function or a substituent borne by the precursor of the substituent —R′₁,
Fₐ is an ether, ester or carbamate function,
the degree of substitution of the saccharide units, i, with —R′₁, being between 0 and 6-j, 0≦i≦6-j and,
Fₐ and Fₐ′ are identical or different,
Fₐ and Gₐ are identical or different,
i+j≦6,
—R′₁ identical to or different than —R₁—,
the free salifiable acid functions borne by the substituent —R′₁ are in the form of alkali metal cation salts,
said identical or different glycosidic bonds being chosen from the group consisting of glycosidic bonds of (1,1), (1,2), (1,3), (1,4) or (1,6) type, in an alpha or beta geometry.

In one embodiment, the anionic compounds according to the invention are characterized in that the radical -[AA]- is derived from an alpha-amino acid.

In one embodiment, the anionic compounds according to the invention are characterized in that the alpha-amino acid is chosen from the group comprising alpha-methylphenylalanine, alpha-methyltyrosine, O-methyltyrosine, alpha-phenylglycine, 4-hydroxyphenylglycine and 3,5-dihydroxyphenylglycine, in their L, D or racemic forms.

In one embodiment, the anionic compounds according to the invention are characterized in that the alpha-amino acid is chosen from natural alpha-amino acids.

In one embodiment, the anionic compounds according to the invention are characterized in that the natural alpha-amino acid is chosen from hydrophobic amino acids chosen from the group comprising tryptophan, leucine, alanine, isoleucine, glycine, phenylalanine, tyrosine and valine, in their L, D or racemic forms.

In one embodiment, the anionic compounds according to the invention are characterized in that the natural alpha-amino acid is chosen from polar amino acids chosen from the group comprising aspartic acid, glutamic acid, lysine, serine and threonine, in their L, D or racemic forms.

In one embodiment, the substituted anionic compounds are characterized in that they are chosen from the anionic compounds substituted with substituents of formula I, II or V in which a is equal to 0.

In one embodiment, the substituted anionic compounds are characterized in that they are chosen from the anionic compounds substituted with substituents of formula I in which G is an ester function.

In one embodiment, the substituted anionic compounds are characterized in that they are chosen from the anionic compounds substituted with substituents of formula I in which G is an amide function.

In one embodiment, the substituted anionic compounds are characterized in that they are chosen from the anionic compounds substituted with substituents of formula I in which G is a carbamate function.

In one embodiment, the substituted anionic compounds are characterized in that they are chosen from the anionic compounds substituted with substituents of formula II in which G′ is an ester function.

In one embodiment, the substituted anionic compounds are characterized in that they are chosen from the anionic compounds substituted with substituents of formula II in which G′ is an amide function.

In one embodiment, the substituted anionic compounds are characterized in that they are chosen from the anionic compounds substituted with substituents of formula II in which G′ is a carbamate function.

In one embodiment, the substituted anionic compounds are characterized in that they are chosen from the anionic compounds substituted with substituents of formula I, II or V in which a is equal to 1.

In one embodiment, the substituted anionic compounds are characterized in that they are chosen from the anionic compounds substituted with substituents of formula I or II in which F is an ether function.

In one embodiment, the substituted anionic compounds are characterized in that they are chosen from the anionic compounds substituted with substituents of formula I or II in which F is an ester function.

In one embodiment, the substituted anionic compounds are characterized in that they are chosen from the anionic compounds substituted with substituents of formula I or II in which F is an amide function.

In one embodiment, the substituted anionic compounds are characterized in that they are chosen from the anionic compounds substituted with substituents of formula I or II in which F is a carbamate function.

In one embodiment, the substituted anionic compounds are characterized in that they are chosen from the anionic compounds substituted with substituents of formula V in which Fₐ is an ether function.

In one embodiment, the substituted anionic compounds are characterized in that they are chosen from the anionic compounds substituted with substituents of formula V in which Fₐ is an ester function.

In one embodiment, the substituted anionic compounds are characterized in that they are chosen from the anionic compounds substituted with substituents of formula V in which Fₐ is a carabamate function.

In one embodiment, the substituted anionic compounds are characterized in that they are chosen from the anionic compounds substituted with substituents of formula I in which T is an amide function.

In one embodiment, the substituted anionic compounds are characterized in that they are chosen from the anionic compounds substituted with substituents of formula I in which T is an ester function.

In one embodiment, the substituted anionic compounds are characterized in that they are chosen from the anionic compounds substituted with substituents of formula I in which T is an amide function, and F is an ether function.

In one embodiment, the substituted anionic compounds are characterized in that they are chosen from the anionic compounds substituted with substituents of formula I in which T is an amide function, and F is an ester function.

In one embodiment, the substituted anionic compounds are characterized in that they are chosen from the anionic compounds substituted with substituents of formula I in which T is an amide function, and F is a carbamate function.

In one embodiment, the substituted anionic compounds are characterized in that they are chosen from the anionic compounds substituted with substituents of formula I in which T is an amide function, and F is an amide function.

In one embodiment, the substituted anionic compounds are characterized in that they are chosen from the anionic compounds substituted with substituents of formula I in which T is an ester function, and F is an ether function.

In one embodiment, the substituted anionic compounds are characterized in that they are chosen from the anionic compounds substituted with substituents of formula I in which T is an ester function, and F is an ester function.

In one embodiment, the substituted anionic compounds are characterized in that they are chosen from the anionic compounds substituted with substituents of formula I in which T is an ester function, and F is a carabamate function.

In one embodiment, the substituted anionic compounds are characterized in that they are chosen from the anionic compounds substituted with substituents of formula I in which T is an ester function, and F is an amide function.

In one embodiment, the substituted anionic compounds are characterized in that they are chosen from the anionic compounds substituted with substituents of formula I or II in which F′ is an ether function.

In one embodiment, the substituted anionic compounds are characterized in that they are chosen from the anionic compounds substituted with substituents of formula I or II in which F′ is an ester function.

In one embodiment, the substituted anionic compounds are characterized in that they are chosen from the anionic compounds substituted with substituents of formula I or II in which F′ is an amide function.

In one embodiment, the substituted anionic compounds are characterized in that they are chosen from the anionic compounds substituted with substituents of formula I or II in which F′ is a carbamate function.

In one embodiment, the substituted anionic compounds are characterized in that they are chosen from the anionic compounds substituted with substituents of formula I or II in which Fₐ is an ether function.

In one embodiment, the substituted anionic compounds are characterized in that they are chosen from the anionic compounds substituted with substituents of formula I or II in which Fₐ is an ester function.

In one embodiment, the substituted anionic compounds are characterized in that they are chosen from the anionic compounds substituted with substituents of formula I or II in which Fₐ is a carbamate function.

In one embodiment, the substituted anionic compounds are characterized in that they are chosen from the anionic compounds substituted with substituents of formula I or II in which Fₐ′ is an ether function.

In one embodiment, the substituted anionic compounds are characterized in that they are chosen from the anionic compounds substituted with substituents of formula I or II in which Fₐ′ is an ester function.

In one embodiment, the substituted anionic compounds are characterized in that they are chosen from the anionic compounds substituted with substituents of formula I or II in which Fₐ′ is a carbamate function.

In one embodiment, the substituted anionic compounds are characterized in that they are chosen from the anionic compounds substituted with substituents of formula I or II in which F and F′ are identical.

In one embodiment, the substituted anionic compounds are characterized in that they are chosen from the anionic compounds substituted with substituents of formula I or II in which F and F′ are ether functions.

In one embodiment, the substituted anionic compounds are characterized in that they are chosen from the anionic compounds substituted with substituents of formula I or II in which F and F′ are ester functions.

In one embodiment, the substituted anionic compounds are characterized in that they are chosen from the anionic compounds substituted with substituents of formula I or II in which F and F′ are amide functions.

In one embodiment, the substituted anionic compounds are characterized in that they are chosen from the anionic compounds substituted with substituents of formula I or II in which F and F′ are carabamate functions.

In one embodiment, the substituted anionic compounds are characterized in that they are chosen from the anionic compounds substituted with substituents of formula I or II or V in which, when the radical —R₁— is a carbon-based chain, it optionally comprises a heteroatom chosen from the group consisting of O, N and S.

In one embodiment, the substituted anionic compounds are characterized in that they are chosen from the anionic compounds substituted with substituents of formula I or II or V in which the radical —R₁— is chosen from the radicals of formulae III and IV below:

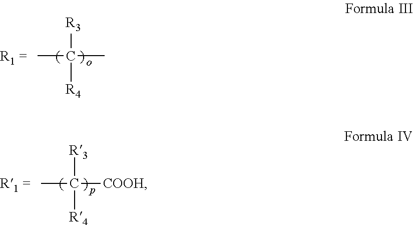

in which:
o and p, which may be identical or different, are greater than or equal to 1 and less than or equal to 12, and
R₃, R′₃, R₄ and R′₄, which may be identical or different, are chosen from the group consisting of a hydrogen atom, a saturated or unsaturated, linear, branched or cyclic C₁ to C₆ alkyl, a benzyl, and a C₇ to C₁₀ alkyl-aryl and optionally comprising heteroatoms chosen from the group consisting of O, N and/or S, or functions chosen from the group consisting of carboxylic acid, amine, alcohol or thiol functions.

In one embodiment, the substituted anionic compounds are characterized in that they are chosen from the anionic compounds substituted with substituents of formula I or II or V in which the radical —R₁—, before attachment to the radical -[AA]- or to the radical -[Q]-, is —CH₂—COOH, and after attachment is —CH₂—.

In one embodiment, the substituted compounds are characterized in that they are chosen from the anionic compounds substituted with substituents of formula I or II or V in which the radical —R₁—, before attachment to the radical -[AA]- or to the radical -[Q]-, is a C₂ to C₁₀ carbon-based chain bearing a carboxylic acid group and, after attachment, is a C₂ to C₁₀ carbon-based chain.

In one embodiment, the substituted compounds are characterized in that they are chosen from the anionic compounds substituted with substituents of formula I or II or V in which the radical —R₁—, before attachment to the radical -[AA]- or to the radical -[Q]-, is a C₂ to C₁₀ carbon-based chain bearing a carboxylic acid group and, after attachment, is a C₂ to C₁₀ carbon-based chain.

In one embodiment, the substituted compounds are characterized in that they are chosen from the anionic compounds substituted with substituents of formula I or II or V in which the radical —R₁—, before attachment to the radical -[AA]- or to the radical -[Q]-, is a C₂ to C₅ carbon-based chain bearing a carboxylic acid group and, after attachment, is a C₂ to C₅ carbon-based chain.

In one embodiment, the substituted compounds are characterized in that they are chosen from the anionic compounds substituted with substituents of formula I or II or V in which the radical —R₁—, before attachment to the radical -[AA]- or to the radical -[Q]-, is a C₂ to C₅ carbon-based chain bearing a carboxylic acid group and, after attachment, is a C₂ to C₅ carbon-based chain.

In one embodiment, the substituted anionic compounds are characterized in that they are chosen from the anionic compounds substituted with substituents of formula I or II or V in which the radical —R₁—, before attachment to the radical -[AA]- or to the radical -[Q]-, is chosen from the following groups, in which * represents the site of attachment to F:

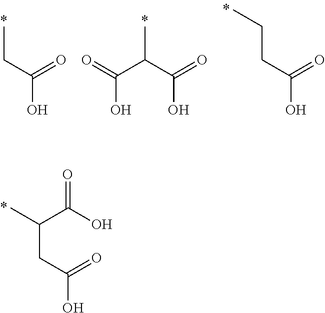

or their salts of alkali metal cations chosen from the group consisting of Na⁺ or K⁺.

In one embodiment, the substituted anionic compounds are characterized in that they are chosen from the anionic compounds substituted with substituents of formula I or II or V in which the radical —R₁—, before attachment to the radical -[AA]- or to the radical -[Q]-, is derived from citric acid.

In one embodiment, the substituted anionic compounds are characterized in that they are chosen from the anionic compounds substituted with substituents of the formula I or II or V in which the radical —R₁—, before attachment to the radical -[AA]- or to the radical -[Q]-, is derived from malic acid.

In one embodiment, the substituted anionic compounds are characterized in that they are chosen from the anionic compounds substituted with substituents of formula I or II or V and do not bear a substituent —R′₁.

In one embodiment, the substituted anionic compounds are characterized in that they are chosen from the anionic compounds substituted with substituents of formula I or II or V in which, when the substituent —R′₁ is a carbon-based chain, it optionally comprises a heteroatom chosen from the group consisting of O, N and S.

In one embodiment, the substituted anionic compounds are characterized in that they are chosen from the anionic compounds substituted with substituents of formula I or II or V in which the substituent —R′₁ is chosen from the radicals of formulae III and IV below:

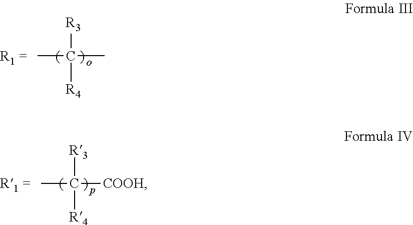

in which:
o and p, which may be identical or different, are greater than or equal to 1 and less than or equal to 12, and
R₃, R′₃, R₄ and R′₄, which may be identical or different, are chosen from the group consisting of a hydrogen atom, a saturated or unsaturated, linear, branched or cyclic C₁ to C₆ alkyl, a benzyl and an alkyl-aryl and optionally comprising heteroatoms chosen from the group consisting of O, N and/or S, or functions chosen from the group consisting of carboxylic acid, amine, alcohol or thiol functions.

In one embodiment, the substituted compounds are characterized in that they are chosen from the anionic compounds substituted with substituents of formula I or II or V in which the substituent —R′₁ is —CH₂COOH.

In one embodiment, the substituted compounds are characterized in that they are chosen from the anionic compounds substituted with substituents of formula I or II or V in which the radical —R′₁—, before attachment to the radical -[AA]- or to the radical -[Q]-, is a C₂ to C₁₀ carbon-based chain bearing a carboxylic acid group and after attachment is a C₂ to C₁₀ carbon-based chain.

In one embodiment, the substituted compounds are characterized in that they are chosen from the anionic compounds substituted with substituents of formula I or II or V in which the radical —R′₁—, before attachment to the radical -[AA]- or to the radical -[Q]-, is a C₂ to C₁₀ carbon-based chain bearing a carboxylic acid group and after attachment is a C₂ to C₁₀ carbon-based chain.

In one embodiment, the substituted compounds are characterized in that they are chosen from the anionic compounds substituted with substituents of formula I or II or V in which the radical —R′₁—, before attachment to the radical -[AA]- or to the radical -[Q]-, is a C₂ to C₅ carbon-based chain bearing a carboxylic acid group and after attachment is a C₂ to C₅ carbon-based chain.

In one embodiment, the substituted compounds are characterized in that they are chosen from the anionic compounds substituted with substituents of formula I or II or V in which the radical —R′₁—, before attachment to the radical -[AA]- or to the radical -[Q]-, is a C₂ to C₅ carbon-based chain bearing a carboxylic acid group and after attachment is a C₂ to C₅ carbon-based chain.

In one embodiment, the substituted anionic compounds are characterized in that they are chosen from the anionic compounds substituted with substituents of formula I or II in which the substituent —R′₁ is chosen from the following groups, in which * represents the site of attachment to Fₐ:

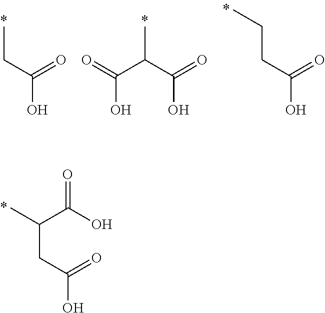

or their salts of alkali metal cations chosen from the group consisting of Na⁺ or K⁺.

In one embodiment, the substituted anionic compounds are characterized in that they are chosen from the anionic compounds substituted with substituents of formula V in which the substituent —R′₁ is chosen from the following groups, in which * represents the site of attachment to Fₐ:

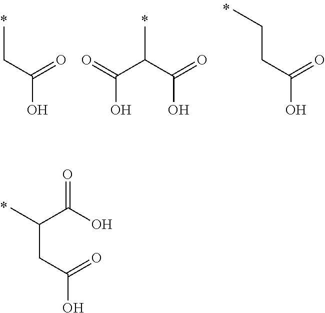

or their salts of alkali metal cations chosen from the group consisting of Na⁺ or K⁺.

In one embodiment, the substituted anionic compounds are characterized in that they are chosen from the anionic compounds substituted with substituents of formula I or II or V in which the substituent —R′₁ is derived from citric acid.

In one embodiment, the substituted anionic compounds are characterized in that they are chosen from the anionic compounds substituted with substituents of formula I or II or V in which the substituent is derived from malic acid.

In one embodiment, the substituted anionic compounds are characterized in that they are chosen from the anionic compounds substituted with substituents of formula I in which Z is an ester function.

In one embodiment, the substituted anionic compounds are characterized in that they are chosen from the anionic compounds substituted with substituents of formula I in which Z is an amide function.

In one embodiment, the substituted anionic compounds are characterized in that they are chosen from the anionic compounds substituted with substituents of formula I in which Z is a carbamate function.

In one embodiment, the substituted anionic compounds are characterized in that they are chosen from the anionic compounds substituted with substituents of formula II in which Z′ is an ester function.

In one embodiment, the substituted anionic compounds are characterized in that they are chosen from the anionic compounds substituted with substituents of formula II in which Z′ is an amide function.

In one embodiment, the substituted anionic compounds are characterized in that they are chosen from the anionic compounds substituted with substituents of formula II in which Z′ is a carbamate function.

In one embodiment, the substituted anionic compounds are characterized in that they are chosen from the anionic compounds substituted with substituents of formula I in which Z is an ester function, T is an amide function, and F is an ether function.

In one embodiment, the substituted anionic compounds are characterized in that they are chosen from the anionic compounds substituted with substituents of formula I in which Z is an ester function, T is an amide function, and F is an ester function.

In one embodiment, the substituted anionic compounds are characterized in that they are chosen from the anionic compounds substituted with substituents of formula I in which Z is an ester function, T is an amide function, and F is a carbamate function.

In one embodiment, the substituted anionic compounds are characterized in that they are chosen from the anionic compounds substituted with substituents of formula I in which Z is an ester function, T is an amide function, and F is an amide function.

In one embodiment, the substituted anionic compounds are characterized in that they are chosen from the anionic compound substituted with substituents of formula I in which Z is an ester function, T is an ester function, and F is an ether function.

In one embodiment, the substituted anionic compounds are characterized in that they are chosen from the anionic compound substituted with substituents of formula I in which Z is an ester function, T is an ester function, and F is an ester function.

In one embodiment, the substituted anionic compounds are characterized in that they are chosen from the anionic compound substituted with substituents of formula I in which Z is an ester function, T is an ester function, and F is a carbamate function.

In one embodiment, the substituted anionic compounds are characterized in that they are chosen from the anionic compound substituted with substituents of formula I in which Z is an ester function, T is an ester function, and F is an amide function.

In one embodiment, the substituted anionic compounds are characterized in that they are chosen from the anionic compounds substituted with substituents of formula I in which Z is an amide function, T is an amide function, and F is an ether function.

In one embodiment, the substituted anionic compounds are characterized in that they are chosen from the anionic compounds substituted with substituents of formula I in which Z is an amide function, T is an amide function, and F is an ester function.

In one embodiment, the substituted anionic compounds are characterized in that they are chosen from the anionic compounds substituted with substituents of formula I in which Z is an amide function, T is an amide function, and F is a carbamate function.

In one embodiment, the substituted anionic compounds are characterized in that they are chosen from the anionic compounds substituted with substituents of formula I in which Z is an amide function, T is an amide function, and F is an amide function.

In one embodiment, the substituted anionic compounds are characterized in that they are chosen from the anionic compound substituted with substituents of formula I in which Z is an amide function, T is an ester function, and F is an ether function.

In one embodiment, the substituted anionic compounds are characterized in that they are chosen from the anionic compound substituted with substituents of formula I in which Z is an amide function, T is an ester function, and F is an ester function.

In one embodiment, the substituted anionic compounds are characterized in that they are chosen from the anionic compound substituted with substituents of formula I in which Z is an amide function, T is an ester function, and F is a carbamate function.

In one embodiment, the substituted anionic compounds are characterized in that they are chosen from the anionic compound substituted with substituents of formula I in which Z is an amide function, T is an ester function, and F is an amide function.

In one embodiment, the substituted anionic compounds are characterized in that they are chosen from the anionic compounds substituted with substituents of formula I in which Z is a carbamate function, T is an amide function, and F is an ether function.

In one embodiment, the substituted anionic compounds are characterized in that they are chosen from the anionic compounds substituted with substituents of formula I in which Z is a carbamate function, T is an amide function, and F is an ester function.

In one embodiment, the substituted anionic compounds are characterized in that they are chosen from the anionic compounds substituted with substituents of formula I in which Z is a carbamate function, T is an amide function, and F is a carbamate function.

In one embodiment, the substituted anionic compounds are characterized in that they are chosen from the anionic compounds substituted with substituents of formula I in which Z is a carbamate function, T is an amide function, and F is an amide function.

In one embodiment, the substituted anionic compounds are characterized in that they are chosen from the anionic compounds substituted with substituents of formula I in which Z is a carbamate function, T is an ester function, and F is an ether function.

In one embodiment, the substituted anionic compounds are characterized in that they are chosen from the anionic compounds substituted with substituents of formula I in which Z is a carbamate function, T is an ester function, and F is an ester function.

In one embodiment, the substituted anionic compounds are characterized in that they are chosen from the anionic compounds substituted with substituents of formula I in which Z is a carbamate function, T is an ester function, and F is a carbamate function.

In one embodiment, the substituted anionic compounds are characterized in that they are chosen from the anionic compounds substituted with substituents of formula I in which Z is a carbamate function, T is an ester function, and F is an amide function.

In one embodiment, the substituted anionic compounds are characterized in that they are chosen from the anionic compounds substituted with substituents of formula I in which G is an ester function and Z is an ester function.

In one embodiment, the substituted anionic compounds are characterized in that they are chosen from the anionic compounds substituted with substituents of formula I in which G is an amide function and Z is an ester function.

In one embodiment, the substituted anionic compounds are characterized in that they are chosen from the anionic compounds substituted with substituents of formula I in which G is a carbamate function and Z is an ester function.

In one embodiment, the substituted anionic compounds are characterized in that they are chosen from the anionic compounds substituted with substituents of formula I in which G is an ester function and Z is an amide function.

In one embodiment, the substituted anionic compounds are characterized in that they are chosen from the anionic compounds substituted with substituents of formula I in which G is an amide function and Z is an amide function.

In one embodiment, the substituted anionic compounds are characterized in that they are chosen from the anionic compounds substituted with substituents of formula I in which G is a carbamate function and Z is an amide function.

In one embodiment, the substituted anionic compounds are characterized in that they are chosen from the anionic compounds substituted with substituents of formula I in which G is an ester function and Z is a carbamate function.

In one embodiment, the substituted anionic compounds are characterized in that they are chosen from the anionic compounds substituted with substituents of formula I in which G is an amide function and Z is a carbamate function.

In one embodiment, the substituted anionic compounds are characterized in that they are chosen from the anionic compounds substituted with substituents of formula I in which G is a carbamate function and Z is a carbamate function.

In one embodiment, the substituted anionic compounds are characterized in that they are chosen from the anionic compounds substituted with substituents of formula II in which G′ is an ester function and Z′ is an ester function.

In one embodiment, the substituted anionic compounds are characterized in that they are chosen from the anionic compounds substituted with substituents of formula II in which G′ is an amide function and Z′ is an ester function.

In one embodiment, the substituted anionic compounds are characterized in that they are chosen from the anionic compounds substituted with substituents of formula II in which G′ is a carbamate function and Z′ is an ester function.

In one embodiment, the substituted anionic compounds are characterized in that they are chosen from the anionic compounds substituted with substituents of formula II in which G′ is an ester function and Z′ is an amide function.

In one embodiment, the substituted anionic compounds are characterized in that they are chosen from the anionic compounds substituted with substituents of formula II in which G′ is an amide function and Z′ is an amide function.

In one embodiment, the substituted anionic compounds are characterized in that they are chosen from the anionic compounds substituted with substituents of formula II in which G′ is a carbamate function and Z′ is an amide function.

In one embodiment, the substituted anionic compounds are characterized in that they are chosen from the anionic compounds substituted with substituents of formula II in which G′ is an ester function and Z′ is a carbamate function.

In one embodiment, the substituted anionic compounds are characterized in that they are chosen from the anionic compounds substituted with substituents of formula II in which G′ is an amide function and Z′ is a carbamate function.

In one embodiment, the substituted anionic compounds are characterized in that they are chosen from the anionic compounds substituted with substituents of formula II in which G′ is a carbamate function and Z′ is a carbamate function.

In one embodiment, the substituted anionic compounds are characterized in that they are chosen from the anionic compounds substituted with substituents of formula I or II in which the radical —R₂ is a benzyl radical.

In one embodiment, the substituted anionic compounds are characterized in that they are chosen from the anionic compounds substituted with substituents of formula I or II in which the radical —R₂ is derived from a hydrophobic alcohol.

In one embodiment, the anionic compounds according to the invention are characterized in that the hydrophobic alcohol is chosen from alcohols consisting of an unsaturated and/or saturated, branched or unbranched alkyl chain comprising from 4 to 18 carbon atoms.

In one embodiment, the anionic compounds according to the invention are characterized in that the hydrophobic alcohol is chosen from alcohols consisting of an unsaturated and/or saturated, branched or unbranched alkyl chain comprising from 6 to 18 carbon atoms.

In one embodiment, the anionic compounds according to the invention are characterized in that the hydrophobic alcohol is chosen from alcohols consisting of an unsaturated and/or saturated, branched or unbranched alkyl chain comprising from 8 to 16 carbon atoms.

In one embodiment, the anionic compounds according to the invention are characterized in that the hydrophobic alcohol is octanol.

In one embodiment, the anionic compounds according to the invention are characterized in that the hydrophobic alcohol is 2-ethylbutanol.

In one embodiment, the anionic compounds according to the invention are characterized in that the hydrophobic alcohol is chosen from myristyl alcohol, cetyl alcohol, stearyl alcohol, cetearyl alcohol, butyl alcohol and oleyl alcohol.

In one embodiment, the anionic compounds according to the invention are characterized in that the hydrophobic alcohol is chosen from the group consisting of cholesterol and its derivatives.

In one embodiment, the anionic compounds according to the invention are characterized in that the hydrophobic alcohol is cholesterol.

In one embodiment, the anionic compounds according to the invention are characterized in that the hydrophobic alcohol is chosen from menthol derivatives.

In one embodiment, the anionic compounds according to the invention are characterized in that the hydrophobic alcohol is menthol in its racemic form.

In one embodiment, the anionic compounds according to the invention are characterized in that the hydrophobic alcohol is the D isomer of menthol.

In one embodiment, the anionic compounds according to the invention are characterized in that the hydrophobic alcohol is the L isomer of menthol.

In one embodiment, the anionic compounds according to the invention are characterized in that the hydrophobic alcohol is chosen from tocopherols.

In one embodiment, the anionic compounds according to the invention are characterized in that the tocopherol is alpha-tocopherol.

In one embodiment, the anionic compounds according to the invention are characterized in that the alpha-tocopherol is the racemate of alpha-tocopherol.

In one embodiment, the anionic compounds according to the invention are characterized in that the tocopherol is the D isomer of alpha-tocopherol.

In one embodiment, the anionic compounds according to the invention are characterized in that the tocopherol is the L isomer of alpha tocopherol.

In one embodiment, the anionic compounds according to the invention are characterized in that the hydrophobic alcohol is chosen from alcohols bearing an aryl group.

In one embodiment, the anionic compounds according to the invention are characterized in that the alcohol bearing an aryl group is chosen from the group consisting of benzyl alcohol and phenethyl alcohol.

In one embodiment, the substituted anionic compounds are characterized in that they are chosen from the anionic compounds substituted with substituents of formula I or II in which the radical —R₂ is derived from a hydrophobic acid.

In one embodiment, the anionic compounds according to the invention are characterized in that the hydrophobic acid is chosen from fatty acids.

In one embodiment, the anionic compounds according to the invention are characterized in that the fatty acids are chosen from the group consisting of acids consisting of a saturated or unsaturated, branched or unbranched alkyl chain comprising from 6 to 30 carbon atoms.

In one embodiment, the anionic compounds according to the invention are characterized in that the fatty acids are chosen from the group consisting of linear fatty acids.

In one embodiment, the anionic compounds according to the invention are characterized in that the linear fatty acids are chosen from the group consisting of caproic acid, enanthic acid, caprylic acid, capric acid, nonanoic acid, decanoic acid, undecanoic acid, dodecanoic acid, palmitic acid, stearic acid, arachidic acid, behenic acid, tricosanoic acid, lignoceric acid, heptacosanoic acid, octacosanoic acid and melissic acid.

In one embodiment, the anionic compounds according to the invention are characterized in that the fatty acids are chosen from the group consisting of unsaturated fatty acids.

In one embodiment, the anionic compounds according to the invention are characterized in that the unsaturated fatty acids are chosen from the group consisting of myristoleic acid, palmitoleic acid, oleic acid, elaidic acid, linoleic acid, alpha-linoleic acid, arachidonic acid, eicosapentaenoic acid, erucic acid and docosahexaenoic acid.

In one embodiment, the anionic compounds according to the invention are characterized in that the fatty acids are chosen from the group consisting of bile acids and their derivatives.

In one embodiment, the anionic compounds according to the invention are characterized in that the bile acids and their derivatives are chosen from the group consisting of cholic acid, dehydrocholic acid, deoxycholic acid and chenodeoxycholic acid.

In one embodiment, the substituted anionic compounds are characterized in that they are chosen from the anionic compounds substituted with substituents of formula II in which n is equal to 0, and the radical —R₁— and the substituent which are identical, are carbon-based chains.

In one embodiment, the substituted anionic compounds are characterized in that they are chosen from the anionic compounds substituted with substituents of formula II in which n is equal to 0, and the radical -[AA]- is an amino acid residue.

In one embodiment, the substituted anionic compounds are characterized in that they are chosen from the anionic compounds substituted with substituents of formula II in which n is equal to 0, the radical —R₁— and the substituent —R′₁, which are identical, are carbon-based chains and the radical -[AA]- is a phenylalanine residue.

In one embodiment, the substituted anionic compounds are characterized in that they are chosen from the anionic compounds substituted with substituents of formula II in which n is equal to 0, the radical —R₁— and the substituent —R′₁, which are identical, are carbon-based chains bonded to the backbone via an ether function and the radical -[AA]- is a phenylalanine residue.

In one embodiment, the substituted anionic compounds are characterized in that they are chosen from the anionic compounds substituted with substituents of formula II in which n is equal to 0, the radical —R₁— and the substituent —R′₁, which are identical, are carbon-based chains bonded to the backbone via a carbamate function and the radical -[AA]- is a phenylalanine residue.

In one embodiment, the substituted anionic compounds are characterized in that they are chosen from the anionic compounds substituted with substituents of formula II in which n is equal to 0, the radical —R₁— and the substituent —R′₁, which are identical, are carbon-based chains bonded to the backbone via an ether function and the radical -[AA]- is a tryptophan residue.

In one embodiment, the substituted anionic compounds are characterized in that they are chosen from the anionic compounds substituted with substituents of formula II in which n is equal to 0, the radical —R₁— and the substituent —R′₁, which are identical, are carbon-based chains bonded to the backbone via an ether function and the radical -[AA]- is a leucine residue.

In one embodiment, the substituted anionic compounds are characterized in that they are chosen from the anionic compounds substituted with substituents of formula II in which n is equal to 0, the radical —R₁— and the substituent —R′₁, which are identical, are carbon-based chains bonded to the backbone via an ether function and the radical -[AA]- is an alpha-phenylglycine residue.

In one embodiment, the substituted anionic compounds are characterized in that they are chosen from the anionic compounds substituted with substituents of formula II in which n is equal to 0, the radical —R₁— and the substituent —R′₁, which are identical, are carbon-based chains bonded to the backbone via an ether function and the radical -[AA]- is a tyrosine residue.

In one embodiment, the substituted anionic compounds are characterized in that they are chosen from the anionic compounds substituted with substituents of formula II in which n and a are equal to 0.

In one embodiment, the substituted anionic compounds are characterized in that they are chosen from the anionic compounds substituted with substituents of formula II in which n and a are equal to 0 and the radical -[AA]- is a phenylalanine residue directly bonded to the backbone via a carbamate function.

In one embodiment, the substituted anionic compounds are characterized in that they are chosen from the anionic compounds substituted with substituents of formula I in which n is equal to 1, and the radical —R₁— and the substituent —R′₁, which are identical, are carbon-based chains.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains and the radical -[Q]- is derived from a diamine.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains, the radical -[Q]- is derived from a diamine and the radical —R₂ is derived from a linear fatty acid.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from a diamine and the radical —R₂ is derived from a linear fatty acid.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from ethylenediamine and the radical —R₂ is derived from a linear fatty acid.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from ethylenediamine and the radical —R₂ is derived from dodecanoic acid.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from a diamine and the radical —R₂ is derived from a hydrophobic alcohol.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from a diamine and the radical —R₂ is derived from cholesterol.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from ethylenediamine and the radical —R₂ is derived from cholesterol.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains, the radical -[Q]- is derived from an amino alcohol and the radical —R₂ is derived from a linear fatty acid.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from an amino alcohol and the radical —R₂ is derived from a linear fatty acid.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from ethanolamine and the radical —R₂ is derived from a linear fatty acid.

In one embodiment, the substituted anionic compounds are characterized in that they are chosen from the anionic compounds substituted with substituents of formula I in which n is equal to 1, the radical —R₁— and the substituent —R′₁, which are identical, are carbon-based chains bonded to the backbone via an ether function, the radical -[Q]- is derived from ethanolamine and the radical —R₂ is derived from dodecanoic acid.

In one embodiment, the substituted anionic compounds are characterized in that they are chosen from the anionic compounds substituted with substituents of formula II in which n is equal to 1, and the radical —R₁— and the substituent —R′₁, which are identical, are carbon-based chains.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains and the radical —R₂ is derived from a linear fatty acid.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains bonded to the backbone via an ether function and the radical —R₂ is derived from a linear fatty acid.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains bonded to the backbone via an ether function, the radical -[AA]- is a lysine residue and the radical —R₂ is derived from a linear fatty acid.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains bonded to the backbone via an ether function, the radical -[AA]- is a lysine residue and the radical —R₂ is derived from dodecanoic acid.

In one embodiment, the substituted anionic compounds are characterized in that they are chosen from the anionic compound substituted with substituents of formula II in which n is equal to 1, the radical —R₁— and the substituent —R′₁, which are identical, are carbon-based chains and the radical —R₂ is derived from a hydrophobic alcohol.

In one embodiment, the substituted anionic compounds are characterized in that they are chosen from the anionic compound substituted with substituents of formula II in which n is equal to 1, the radical —R₁— and the substituent —R′₁, which are identical, are carbon-based chains bonded to the backbone via an ether function and the radical —R₂ is derived from a hydrophobic alcohol.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains bonded to the backbone via an ether function, the radical -[AA]- is a leucine residue and the radical —R₂ is derived from a hydrophobic alcohol.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains bonded to the backbone via an ether function, the radical -[AA]- is a leucine residue and the radical —R₂ is derived from cholesterol.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains bonded to the backbone via an ether function, the radical -[AA]- is an aspartic acid residue and the radical —R₂ is derived from benzyl alcohol.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains bonded to the backbone via an ether function, the radical -[AA]- is a glycine residue and the radical —R₂ is derived from decanol.

In one embodiment, the substituted anionic compounds are characterized in that they are chosen from the anionic compounds substituted with substituents of formula II in which n is equal to 1, the radical —R₁— and the substituent —R′₁, which are identical, are carbon-based chains bonded to the backbone via an ether function, the radical -[AA]- is a phenylalanine residue and the radical —R₂ is derived from 3,7-dimethyloctanol.

In one embodiment, the substituted anionic compounds are characterized in that they are chosen from the anionic compounds substituted with substituents of formula II in which n is equal to 1 and a is equal to 0.

In one embodiment, the substituted anionic compounds are characterized in that they are chosen from the anionic compounds substituted with substituents of formula II in which n is equal to 1 and a is equal to 0 and R₂ is a carbon-based chain.

In one embodiment, the substituted anionic compounds are characterized in that they are chosen from the anionic compounds substituted with substituents of formula II in which n is equal to 1 and a is equal to 0, the radical -[AA]- is a phenylalanine residue directly bonded to the backbone via an amide function and R₂ is a carbon-based chain.

In one embodiment, the substituted anionic compounds are characterized in that they are chosen from the anionic compounds substituted with substituents of formula II in which n is equal to 1 and a is equal to 0, the radical -[AA]- is a phenylalanine residue directly bonded to the backbone via an amide function and R₂ is derived from methanol.

In one embodiment, the substituted anionic compounds are characterized in that they are chosen from the anionic compounds substituted with substituents of formula I in which n is equal to 2, and the radical —R₁— and the substituent —R′₁, which are identical, are carbon-based chains.

In one embodiment, the substituted anionic compounds are characterized in that they are chosen from the anionic compounds substituted with substituents of formula I in which n is equal to 2, the radical —R₁— and the substituent —R′₁, which are identical, are carbon-based chains bonded to the backbone via an ether function and the radical -[Q]- is derived from a diamine coupled to an amino acid.

In one embodiment, the substituted anionic compounds are characterized in that they are chosen from the anionic compounds substituted with substituents of formula I in which n is equal to 2, the radical —R₁— and the substituent —R′₁, which are identical, are carbon-based chains bonded to the backbone via an ether function, the radical -[Q]- is derived from a diamine coupled to an amino acid and the radical R₂ is derived from a linear fatty acid.

In one embodiment, the substituted anionic compounds are characterized in that they are chosen from the anionic compounds substituted with substituents of formula I in which n is equal to 2, the radical —R₁— and the substituent —R′₁, which are identical, are carbon-based chains, the radical -[Q]- is derived from ethylenediamine coupled to an amino acid and the radical R₂ is derived from a linear fatty acid.

In one embodiment, the substituted anionic compounds are characterized in that they are chosen from the anionic compounds substituted with substituents of formula I in which n is equal to 2, the radical —R₁— and the substituent —R′₁, which are identical, are carbon-based chains bonded to the backbone via an ether function, the radical -[Q]- is derived from ethylenediamine coupled to a lysine and the radical R₂ is derived from a linear fatty acid.

In one embodiment, the substituted anionic compounds are characterized in that they are chosen from the anionic compounds substituted with substituents of formula I in which n is equal to 2, the radical —R₁— and the substituent —R′₁, which are identical, are carbon-based chains bonded to the backbone via an ether function, the radical -[Q]- is derived from ethylenediamine coupled to a lysine and the radical R₂ is derived from dodecanoic acid.

In one embodiment, the substituted anionic compounds are characterized in that they are chosen from the anionic compounds substituted with substituents of formula I in which n is equal to 2, the radical —R₁— and the substituent —R′₁, which are identical, are carbon-based chains bonded to the backbone via an ether function, the radical -[Q]- is derived from ethylenediamine coupled to a lysine and the radical R₂ is derived from dodecanoic acid.

In one embodiment, the substituted anionic compounds are characterized in that they are chosen from the anionic compounds substituted with substituents of formula I in which n is equal to 2, the radical —R₁— and the substituent —R′₁, which are identical, are carbon-based chains bonded to the backbone via an ether function, the radical -[Q]- is derived from ethylenediamine coupled to a lysine and the radical R₂ is derived from octanoic acid.

In one embodiment, the substituted anionic compounds are characterized in that they are chosen from the anionic compounds substituted with substituents of formula II in which n is equal to 2, and the radical —R₁— and the substituent —R′₁, which are identical, are carbon-based chains.

In one embodiment, the substituted anionic compounds are characterized in that they are chosen from the anionic compounds substituted with substituents of formula II in which n is equal to 2, the radical —R₁— and the substituent —R′₁, which are identical, are carbon-based chains bonded to the backbone via an ether function and the radical —R₂ is derived from a hydrophobic alcohol.

In one embodiment, the substituted anionic compounds are characterized in that they are chosen from the anionic compounds substituted with substituents of formula II in which n is equal to 2, the radical —R₁— and the substituent —R′₁, which are identical, are carbon-based chains bonded to the backbone via an ether function, the radical -[AA]- is an aspartic acid residue and the radical —R₂ is derived from a hydrophobic alcohol.

In one embodiment, the substituted anionic compounds are characterized in that they are chosen from the anionic compounds substituted with substituents of formula II in which n is equal to 2, the radical —R₁— and the substituent —R′₁, which are identical, are carbon-based chains bonded to the backbone via an ether function, the radical -[AA]- is an aspartic acid residue and the radical —R₂ is derived from dodecanol.

In one embodiment, the substituted anionic compounds are characterized in that they are chosen from the anionic compounds substituted with substituents of formula II in which n is equal 2, the radical —R₁— and the substituent —R′₁, which are identical, are carbon-based chains bonded to the backbone via an ester function and the radical —R₂ is derived from a hydrophobic alcohol.

In one embodiment, the substituted anionic compounds are characterized in that they are chosen from the anionic compounds substituted with substituents of formula II in which n is equal to 2, the radical —R₁— and the substituent —R′₁, which are identical, are carbon-based chains bonded to the backbone via an ester function, the radical -[AA]- is an aspartic acid residue and the radical —R₂ is derived from a hydrophobic alcohol.

In one embodiment, the substituted anionic compounds are characterized in that they are chosen from the anionic compounds substituted with substituents of formula II in which n is equal to 2, the radical —R₁— and the substituent —R′₁, which are identical, are carbon-based chains bonded to the backbone via an ester function, the radical -[AA]- is an aspartic acid residue and the radical —R₂ is derived from dodecanol.

In one embodiment, the substituted anionic compound in isolated form bears a substituent of general formula I or II or V.

In one embodiment, the substituted anionic compound in isolated form bears two substituents of general formula I or II or V.

In one embodiment, the substituted anionic compound in isolated form bears three substituents of general formula I or II or V.

In one embodiment, the substituted anionic compound in isolated form bears four substituents of general formula I or II or V.

In one embodiment, the substituted anionic compound in isolated form bears five substituents of general formula I or II or V.

In one embodiment, the substituted anionic compound in isolated form bears six substituents of general formula I or II or V.

In one embodiment, the substituted anionic compound in isolated form bears one substituent of general formula I or II or V per saccharide unit.

In one embodiment, the substituted anionic compound in isolated form bears two substituents of general formula I or II or V per saccharide unit.

In one embodiment, the substituted anionic compound in isolated form bears three substituents of general formula I or II or V per saccharide unit.

In one embodiment, the substituted anionic compound in isolated form bears four substituents of general formula I or II or V per saccharide unit.

In one embodiment, the anionic compounds according to the invention are characterized in that at least one saccharide unit is in cyclic form.

In one embodiment, the anionic compounds according to the invention are characterized in that at least one saccharide unit is in open reduced or open oxidized form.

In one embodiment, the anionic compounds according to the invention are characterized in that at least one saccharide unit is chosen from the group of pentoses.

In one embodiment, the anionic compounds according to the invention are characterized in that the pentoses are chosen from the group consisting of arabinose, ribulose, xylulose, lyxose, ribose, xylose, deoxyribose, arabitol, xylitol and ribitol.

In one embodiment, the anionic compounds according to the invention are characterized in that at least one saccharide unit is chosen from the group of hexoses.

In one embodiment, the anionic compounds according to the invention are characterized in that the hexoses are chosen from the group consisting of mannose, glucose, fructose, sorbose, tagatose, psicose, galactose, allose, altrose, talose, idose, gulose, fucose, fuculose, rhamnose, mannitol, xylitol, sorbitol and galactitol (dulcitol).

In one embodiment, the anionic compounds according to the invention are characterized in that at least one saccharide unit is chosen from the group of uronic acids.

In one embodiment, the anionic compounds according to the invention are characterized in that the uronic acids are chosen from the group consisting of glucuronic acid, iduronic acid, galacturonic acid, gluconic acid, mucic acid, glucaric acid and galactonic acid.

In one embodiment, the anionic compounds according to the invention are characterized in that at least one saccharide unit is an N-acetylhexosamine.

In one embodiment, the anionic compounds according to the invention are characterized in that the N-acetylhexosamine is chosen from the group consisting of N-acetylgalactosamine, N-acetylglucosamine and N-acetylmannosamine.

In one embodiment, the anionic compounds according to the invention are characterized in that the backbone is made up of a discrete number u=1 of saccharide units.

In one embodiment, the anionic compounds according to the invention are characterized in that the saccharide unit is chosen from the group consisting of hexoses in cyclic form or in open form.

In one embodiment, the anionic compounds according to the invention are characterized in that the saccharide unit is chosen from the group consisting of glucose, mannose, mannitol, xylitol or sorbitol.

In one embodiment, the anionic compounds according to the invention are characterized in that the saccharide unit is chosen from the group consisting of fructose and arabinose.

In one embodiment, the anionic compounds according to the invention are characterized in that the saccharide unit is N-acetylglucosamine.

In one embodiment, the anionic compounds according to the invention are characterized in that the saccharide unit is N-acetylgalactosamine.

In one embodiment, the anionic compounds according to the invention are characterized in that the saccharide unit is chosen from the group consisting of uronic acids.

In one embodiment, the anionic compounds according to the invention are characterized in that the saccharide units are chosen from the group consisting of glucose, mannose, mannitol, xylitol or sorbitol.

In one embodiment, the anionic compounds according to the invention are characterized in that the saccharide units are chosen from the group consisting of fructose and arabinose.

In one embodiment, the anionic compounds according to the invention are characterized in that at least one of the saccharide units is N-acetylglucosamine.

In one embodiment, the anionic compounds according to the invention are characterized in that at least one of the saccharide units is N-acetylgalactosamine.

In one embodiment, the anionic compounds according to the invention are characterized in that the backbone is made up of a discrete number 2≦u≦8 of identical or different saccharide units.

In one embodiment, the anionic compounds according to the invention are characterized in that the identical or different saccharide units, which make up the backbone made up of a discrete number 2≦u≦8 of saccharide units, are chosen from the group of pentoses in cyclic form and/or in open form.

In one embodiment, the anionic compounds according to the invention are characterized in that the identical or different saccharide units, which make up the backbone made up of a discrete number 2≦u≦8 of saccharide units, are chosen from the group of hexoses in cyclic form and/or in open form.

In one embodiment, the anionic compounds according to the invention are characterized in that the identical or different saccharide units, which make up the backbone made up of a discrete number 2≦u≦8 of saccharide units, are chosen from the group consisting of uronic acids in cyclic form and/or in open form.

In one embodiment, the anionic compounds according to the invention are characterized in that the identical or different saccharide units, which make up the backbone made up of a discrete number 2≦u≦8 of saccharide units, are chosen from the group of hexoses and pentoses.

In one embodiment, the anionic compounds according to the invention are characterized in that the identical or different saccharide units, which make up the backbone made up of a discrete number 2≦u≦8 of saccharide units, are chosen from the group of hexoses.

In one embodiment, the anionic compounds according to the invention are characterized in that the identical or different saccharide units, which make up the backbone made up of a discrete number 2≦u≦8 of saccharide units, are hexoses chosen from the group consisting of glucose and mannose.

In one embodiment, the anionic compounds according to the invention are characterized in that the backbone is made up of a discrete number u=2 of identical or different saccharide units.

In one embodiment, the anionic compounds according to the invention are characterized in that the two saccharide units are identical.

In one embodiment, the anionic compounds according to the invention are characterized in that the two saccharide units are different.

In one embodiment, the anionic compounds according to the invention are characterized in that the identical or different saccharide units are chosen from hexoses and/or pentoses and are linked via a glycosidic bond of (1,1) type.

In one embodiment, the anionic compounds according to the invention are characterized in that the identical or different saccharide units are chosen from hexoses and/or pentoses and are linked via a glycosidic bond of (1,2) type.

In one embodiment, the anionic compounds according to the invention are characterized in that the identical or different saccharide units are chosen from hexoses and/or pentoses and are linked via a glycosidic bond of (1,3) type.

In one embodiment, the anionic compounds according to the invention are characterized in that the identical or different saccharide units are chosen from hexoses and/or pentoses and are linked via a glycosidic bond of (1,4) type.

In one embodiment, the anionic compounds according to the invention are characterized in that the identical or different saccharide units are chosen from hexoses and/or pentoses and are linked via a glycosidic bond of (1,6) type.

In one embodiment, the anionic compounds according to the invention are characterized in that they consist of a backbone made up of a discrete number u=2 of identical or different saccharide units chosen from hexoses linked via a glycosidic bond of (1,1) type.

In one embodiment, the anionic compounds according to the invention are characterized in that the backbone made up of a discrete number u=2 of different saccharide units chosen from hexoses and linked via a glycosidic bond of (1,1) type is chosen from the group consisting of trehalose and sucrose.

In one embodiment, the anionic compounds according to the invention are characterized in that they consist of a backbone made up of a discrete number u=2 of identical or different saccharide units chosen from hexoses linked via a glycosidic bond of (1,2) type.

In one embodiment, the anionic compounds according to the invention are characterized in that the backbone made up of a discrete number u=2 of identical or different saccharide units chosen from hexoses linked via a glycosidic bond of (1,2) type is kojibiose.

In one embodiment, the anionic compounds according to the invention are characterized in that they consist of a backbone made up of a discrete number u=2 of identical or different saccharide units chosen from hexoses linked via a glycosidic bond of (1,3) type.

In one embodiment, the anionic compounds according to the invention are characterized in that the backbone made up of a discrete number u=2 of identical or different saccharide units chosen from hexoses linked via a glycosidic bond of (1,3) type is chosen from the group consisting of nigeriose and laminaribiose.

In one embodiment, the anionic compounds according to the invention are characterized in that they consist of a backbone made up of a discrete number u=2 of identical or different saccharide units chosen from hexoses linked via a glycosidic bond of (1,4) type.

In one embodiment, the anionic compounds according to the invention are characterized in that the backbone made up of a discrete number u=2 of identical or different saccharide units chosen from hexoses linked via a glycosidic bond of (1,4) type is chosen from the group consisting of maltose, lactose and cellobiose.

In one embodiment, the anionic compounds according to the invention are characterized in that they consist of a backbone made up of a discrete number u=2 of identical or different saccharide units chosen from hexoses linked via a glycosidic bond of (1,6) type.

In one embodiment, the anionic compounds according to the invention are characterized in that the backbone made up of a discrete number u=2 of identical or different saccharide units chosen from hexoses linked via a glycosidic bond of (1.6) type is chosen from the group consisting of isomaltose, melibiose and gentiobiose.

In one embodiment, the anionic compounds according to the invention are characterized in that the backbone made up of a discrete number u=2 of identical or different saccharide units chosen from hexoses linked via a glycosidic bond of (1,6) type is isomaltose.

In one embodiment, the anionic compounds according to the invention are characterized in that they consist of a backbone made up of a discrete number u=2 of saccharide units of which one is in cyclic form and the other in open reduced form.

In one embodiment, the anionic compounds according to the invention are characterized in that the backbone made up of a discrete number u=2 of saccharide units of which one is in cyclic form and the other in open reduced form is chosen from the group consisting of maltitol and isomaltitol.

In one embodiment, the anionic compounds according to the invention are characterized in that the backbone is made up of a discrete number 3≦u≦8 of identical or different saccharide units.

In one embodiment, the anionic compounds according to the invention are characterized in that at least one of the identical or different saccharide units, which make up the backbone made up of a discrete number 3≦u≦8 of saccharide units, is chosen from the group consisting of hexose and/or pentose units linked via identical or different glycosidic bonds.

In one embodiment, the anionic compounds according to the invention are characterized in that the identical or different saccharide units, which make up the backbone made up of a discrete number 3≦u≦8 of saccharide units, are chosen from hexoses and/or pentoses and are linked via at least one glycosidic bond of (1,2) type.

In one embodiment, the anionic compounds according to the invention are characterized in that the identical or different saccharide units, which make up the backbone made up of a discrete number 3≦u≦8 of saccharide units, are chosen from hexoses and/or pentoses and are linked via at least one glycosidic bond of (1,3) type.

In one embodiment, the anionic compounds according to the invention are characterized in that the identical or different saccharide units, which make up the backbone made up of a discrete number 3≦u≦8 of saccharide units, are chosen from hexoses and/or pentoses and are linked via at least one glycosidic bond of (1,4) type.

In one embodiment, the anionic compounds according to the invention are characterized in that the identical or different saccharide units, which make up the backbone made up of a discrete number 3≦u≦8 of saccharide units, are chosen from hexoses and/or pentoses and are linked via at least one glycosidic bond of (1,6) type.

In one embodiment, the anionic compounds according to the invention are characterized in that the backbone is made up of a discrete number u=3 of identical or different saccharide units.

In one embodiment, the anionic compounds according to the invention are characterized in that they comprise at least one saccharide unit chosen from the group consisting of hexoses in cyclic form and at least one saccharide unit chosen from the group consisting of hexoses in open form.

In one embodiment, the anionic compounds according to the invention are characterized in that the three saccharide units are identical.

In one embodiment, the anionic compounds according to the invention are characterized in that two of the three saccharide units are identical.

In one embodiment, the anionic compounds according to the invention are characterized in that the identical saccharide units are chosen from hexoses, two of which are in cyclic form and one of which is in open reduced form, and which are linked via glycosidic bonds of (1,4) type.

In one embodiment, the anionic compounds according to the invention are characterized in that the identical saccharide units are chosen from hexoses, two of which are in cyclic form and one of which is in open reduced form, and which are linked via glycosidic bonds of (1,6) type.

In one embodiment, the anionic compounds according to the invention are characterized in that the identical or different saccharide units are chosen from hexoses and that the central hexose is linked via a glycosidic bond of (1,2) type and via a glycosidic bond of (1,4) type.

In one embodiment, the anionic compounds according to the invention are characterized in that the identical or different saccharide units are chosen from hexoses and that the central hexose is linked via a glycosidic bond of (1,3) type and via a glycosidic bond of (1,4) type.

In one embodiment, the anionic compounds according to the invention are characterized in that the identical or different saccharide units are chosen from hexoses and that the central hexose is linked via a glycosidic bond of (1,2) type and via a glycosidic bond of (1,6) type.

In one embodiment, the anionic compounds according to the invention are characterized in that the identical or different saccharide units are chosen from hexoses and that the central hexose is linked via a glycosidic bond of (1,2) type and via a glycosidic bond of (1,3) type.

In one embodiment, the anionic compounds according to the invention are characterized in that the identical or different saccharide units are chosen from hexoses and that the central hexose is linked via a glycosidic bond of (1,4) type and via a glycosidic bond of (1,6) type.

In one embodiment, the anionic compounds according to the invention are characterized in that the backbone is erlose.

In one embodiment, the anionic compounds according to the invention are characterized in that the three identical or different saccharide units are hexose units chosen from the group consisting of mannose and glucose.

In one embodiment, the anionic compound according to the invention is characterized in that the backbone is maltotriose.

In one embodiment, the anionic compound according to the invention is characterized in that the backbone is isomaltotriose.

In one embodiment, the anionic compounds according to the invention are characterized in that the backbone is made up of a discrete number u=4 of identical or different saccharide units.

In one embodiment, the anionic compounds according to the invention are characterized in that the four saccharide units are identical.

In one embodiment, the anionic compounds according to the invention are characterized in that three of the four saccharide units are identical.

In one embodiment, the anionic compounds according to the invention are characterized in that the four saccharide units are hexose units chosen from the group consisting of mannose and glucose.

In one embodiment, the anionic compound according to the invention is characterized in that the backbone is maltotetraose.

In one embodiment, the anionic compounds according to the invention are characterized in that the identical or different saccharide unit are chosen from hexoses and that a terminal hexose is linked via a glycosidic bond of (1,2) type and that the others are linked to one another via a glycosidic bond of (1,6) type.

In one embodiment, the anionic compounds according to the invention are characterized in that the identical or different saccharide units are chosen from hexoses and are linked via a glycosidic bond of (1,6) type.

In one embodiment, the anionic compounds according to the invention are characterized in that the backbone is made up of a discrete number u=5 of identical or different saccharide units.

In one embodiment, the anionic compounds according to the invention are characterized in that the five saccharide units are identical.

In one embodiment, the anionic compounds according to the invention are characterized in that the five saccharide units are hexose units chosen from the group consisting of mannose and glucose.

In one embodiment, the anionic compounds according to the invention are characterized in that the identical or different saccharide units are chosen from hexoses and are linked via a glycosidic bond of (1,4) type.

In one embodiment, the anionic compound according to the invention is characterized in that the backbone is maltopentaose.

In one embodiment, the anionic compounds according to the invention are characterized in that the backbone is made up of a discrete number u=6 of identical or different saccharide units.

In one embodiment, the anionic compounds according to the invention are characterized in that the six saccharide units are identical.

In one embodiment, the anionic compounds according to the invention are characterized in that the identical or different saccharide units are chosen from hexoses and are linked via a glycosidic bond of (1,4) type.

In one embodiment, the anionic compounds according to the invention are characterized in that the six identical or different saccharide units are hexose units chosen from the group consisting of mannose and glucose.

In one embodiment, the anionic compound according to the invention is characterized in that the backbone is maltohexaose.

In one embodiment, the anionic compounds according to the invention are characterized in that the backbone is made up of a discrete number u=7 of identical or different saccharide units.

In one embodiment, the anionic compounds according to the invention are characterized in that the seven saccharide units are identical.

In one embodiment, the anionic compounds according to the invention are characterized in that the identical or different saccharide units are chosen from hexoses and are linked via a glycosidic bond of (1,4) type.

In one embodiment, the anionic compounds according to the invention are characterized in that the seven saccharide units are hexose units chosen from the group consisting of mannose and glucose.

In one embodiment, the anionic compound according to the invention is characterized in that the backbone is maltoheptaose.

In one embodiment, the anionic compounds according to the invention are characterized in that the backbone is made up of a discrete number u=8 of identical or different saccharide units.

In one embodiment, the anionic compounds according to the invention are characterized in that the eight saccharide units are identical.

In one embodiment, the anionic compounds according to the invention are characterized in that the identical or different saccharide units are chosen from hexoses and are linked via a glycosidic bond of (1,4) type.

In one embodiment, the anionic compounds according to the invention are characterized in that the eight saccharide units are hexose units chosen from the group consisting of mannose and glucose.

In one embodiment, the anionic compound according to the invention is characterized in that the backbone is maltooctaose.

In one embodiment, the anionic compound comprising a discrete number of saccharide units is a natural compound.

In one embodiment, the anionic compound comprising a discrete number of saccharide units is a synthetic compound.

In one embodiment, the anionic compounds according to the invention are characterized in that they are obtained by enzymatic degradation of a polysaccharide followed by purification.

In one embodiment, the anionic compounds according to the invention are characterized in that they are obtained by chemical degradation of a polysaccharide followed by purification.

In one embodiment, the anionic compounds according to the invention are characterized in that they are obtained chemically, by covalent coupling of lower-molecular-weight precursors.

In one embodiment, the anionic compounds according to the invention are characterized in that the backbone is sophorose.

In one embodiment, the anionic compounds according to the invention are characterized in that they are chosen from the anionic compounds of which the backbone is sucrose.

In one embodiment, the anionic compounds according to the invention are characterized in that they are chosen from the anionic compounds of which the backbone is lactulose.

In one embodiment, the anionic compounds according to the invention are characterized in that they are chosen from the anionic compounds of which the backbone is maltulose.

In one embodiment, the anionic compounds according to the invention are characterized in that they are chosen from the anionic compounds of which the backbone is leucrose.

In one embodiment, the anionic compounds according to the invention are characterized in that they are chosen from the anionic compounds of which the backbone is N-acetyllactosamine.

In one embodiment, the anionic compounds according to the invention are characterized in that they are chosen from the anionic compounds of which the backbone is N-acetylallolactosamine.

In one embodiment, the anionic compounds according to the invention are characterized in that they are chosen from the anionic compounds of which the backbone is rutinose.

In one embodiment, the anionic compounds according to the invention are characterized in that they are chosen from the anionic compounds of which the backbone is isomaltulose.

In one embodiment, the anionic compounds according to the invention are characterized in that they are chosen from the anionic compounds of which the backbone is fucosyllactose.

In one embodiment, the anionic compounds according to the invention are characterized in that they are chosen from the anionic compounds of which the backbone is gentianose.

In one embodiment, the anionic compounds according to the invention are characterized in that they are chosen from the anionic compounds of which the backbone is raffinose.

In one embodiment, the anionic compounds according to the invention are characterized in that they are chosen from the anionic compounds of which the backbone is melezitose.

In one embodiment, the anionic compounds according to the invention are characterized in that they are chosen from the anionic compounds of which the backbone is panose.

In one embodiment, the anionic compounds according to the invention are characterized in that they are chosen from the anionic compounds of which the backbone is kestose.

In one embodiment, the anionic compounds according to the invention are characterized in that they are chosen from the anionic compounds of which the backbone is stachyose.

The nomenclature used hereinafter and in the examples section is a simplified nomenclature which refers back to the precursor of the functionalized compounds.

In one embodiment, an anionic compound according to the invention is sodium maltotriosemethylcarboxylate functionalized with L-phenylalanine for which i=1.0 and j=0.65.

In one embodiment, an anionic compound according to the invention is sodium maltotriosemethylcarboxylate functionalized with L-phenylalanine for which i=0.65 and j=1.0.

In one embodiment, an anionic compound according to the invention is sodium maltotriosemethylcarboxylate functionalized with L-phenylalanine for which i=0.35 and j=0.65.

In one embodiment, an anionic compound according to the invention is sodium maltotriosemethylcarboxylate functionalized with L-tryptophan for which i=0.65 and j=1.0.

In one embodiment, an anionic compound according to the invention is sodium maltotriosemethylcarboxylate functionalized with cholesteryl leucinate for which i=1.56 and j=0.09.

In one embodiment, an anionic compound according to the invention is sodium N-methylcarboxylate mannitol carbamate modified with L-phenylalanine for which i=0.8 and j=3.5.

In one embodiment, an anionic compound according to the invention is sodium N-phenylalaninate mannitol hexacarbamate for which i=0.0 and j=6.0.

In one embodiment, an anionic compound according to the invention is sodium maltotriosemethylcarboxylate functionalized with L-phenylalanine for which i=1.25 and j=0.4.

In one embodiment, an anionic compound according to the invention is sodium maltotriosemethylcarboxylate functionalized with L-phenylalanine for which i=0.8 and j=0.65.

In one embodiment, an anionic compound according to the invention is sodium maltotriosemethylcarboxylate functionalized with L-phenylalanine for which i=2.65 and j=0.65.

In one embodiment, an anionic compound according to the invention is sodium maltopentaosemethylcarboxylate functionalized with L-phenylalanine for which i=1.0 and j=0.75.

In one embodiment, an anionic compound according to the invention is sodium maltooctaosemethylcarboxylate functionalized with L-phenylalanine for which i=1.0 and j=0.65.

In one embodiment, an anionic compound according to the invention is sodium maltotriosemethylcarboxylate functionalized with cholesteryl leucinate for which i=1.76 and j=0.08.

In one embodiment, an anionic compound according to the invention is sodium maltotriosemethylcarboxylate functionalized with cholesteryl leucinate for which i=1.33 and j=0.29.

In one embodiment, an anionic compound according to the invention is sodium maltotriosemethylcarboxylate functionalized with cholesteryl leucinate for which i=3.01 and j=0.29.

In one embodiment, an anionic compound according to the invention is sodium maltopentaosemethylcarboxylate functionalized with cholesteryl leucinate for which i=1.61 and j=0.14.

In one embodiment, an anionic compound according to the invention is sodium maltooctaosemethylcarboxylate functionalized with cholesteryl leucinate for which i=1.11 and j=0.09.

In one embodiment, an anionic compound according to the invention is sodium maltotriosemethylcarboxylate functionalized with β-benzyl aspartate for which i=1.15 and j=0.53.

In one embodiment, an anionic compound according to the invention is sodium maltotriosemethylcarboxylate functionalized with dilauryl aspartate for which i=2.37 and j=0.36.

In one embodiment, an anionic compound according to the invention is sodium maltotriosemethylcarboxylate functionalized with 2-[(2-dodecanoylamino-6-dodecanoylamino)hexanoylamino]ethanamine for which i=2.52 and j=0.21.

In one embodiment, an anionic compound according to the invention is sodium maltotriosemethylcarboxylate functionalized with N-(2-aminoethyl)dodecanamide for which i=1.37 and j=0.27.

In one embodiment, an anionic compound according to the invention is sodium maltotriosesuccinate functionalized with dilauryl aspartate for which i=2.36 and j=0.41.

In one embodiment, an anionic compound according to the invention is sodium maltotriosemethylcarboxylate functionalized with decanoyl glycinate for which i=1.43 and j=0.21.

In one embodiment, an anionic compound according to the invention is sodium maltotriosemethylcarboxylate functionalized with L-leucine for which i=1.06 and j=0.58.

In one embodiment, an anionic compound according to the invention is sodium maltotriosemethylcarboxylate functionalized with cholesteryl 2-aminoethylcarbamate for which i=2.45 and j=0.28.

In one embodiment, an anionic compound according to the invention is sodium maltotriosemethylcarboxylate functionalized with alpha-phenylglycine for which i=1.12 and j=0.52.

In one embodiment, an anionic compound according to the invention is sodium maltotriosemethylcarboxylate functionalized with 2-[(2-octanoylamino-6-octanoylamino)hexanoylamino]ethanamine for which i=1.36 and j=0.28.

In one embodiment, an anionic compound according to the invention is sodium maltotriosemethylcarboxylate functionalized with L-tyrosine for which i=0.83 and j=0.81.

In one embodiment, an anionic compound according to the invention is sodium maltotriosemethylcarboxylate functionalized with 2-aminoethyl dodecanoate for which i=1.37 and j=0.27.

In one embodiment, an anionic compound according to the invention is sodium maltotriosemethylcarboxylate functionalized with 3,7-dimethyloctanoyl phenylalaninate for which i=1.25 and j=0.39.

In one embodiment, an anionic compound according to the invention is sodium hyaluronate tetrasaccharide functionalized with methyl phenylalaninate for which i=0.28 and j=0.22.

In one embodiment, an anionic compound according to the invention is sodium maltotriosemethylcarboxylate functionalized with 2-[(2-decanoylamino-6-decanoyl-amino)hexanoylamino]ethanamine for which i=1.43 and j=0.21.

In one embodiment, an anionic compound according to the invention is sodium maltotriosemethylcarboxylate functionalized with ε-N-dodecanoyl-L-lysine for which i=1.27 and j=0.37.

In one embodiment, an anionic compound according to the invention is sodium N-phenylalaninate mannitol 2,3,4,5-tetracarbamate for which i=0 and j=4.

The invention also relates to the processes for producing substituted anionic compounds, in isolated form or as a mixture, chosen from the anionic compounds substituted with substituents of formula I or II.

In one embodiment, the substituted anionic compounds chosen from the anionic compounds substituted with substituents of formula I or II are characterized in that they can be obtained by random grafting of the substituents onto the saccharide backbone.

In one embodiment, the substituted anionic compounds chosen from the anionic compounds substituted with substituents of formula I or II are characterized in that they can be obtained by grafting the substituents at precise positions on the saccharide units by means of a process which implements steps of protection/deprotection of the alcohol or carboxylic acid groups naturally borne by the backbone. This strategy results in selective, in particular regioselective, grafting of the substituents onto the backbone. The protective groups include, without limitation, those in the textbook described PGM Wuts, et al., Greene's Protective Groups in Organic Synthesis 2007.

The saccharide backbone can be obtained by degradation of a high-molecular-weight polysaccharide. The degradation routes include, without limitation, chemical degradation and/or enzymatic degradation.

The saccharide backbone can also be obtained by formation of glycosidic bonds between monosaccharide or oligosaccharide molecules using an enzymatic or chemical coupling strategy. The coupling strategies include those described in the publication J T Smooth et al., Advances in Carbohydrate Chemistry and Biochemistry 2009, 62, 162-236 and in the textbook T K Lindhorst, Essentials of Carbohydrate Chemistry and Biochemistry 2007, 157-209. The coupling reactions can be carried out in solution or on a solid support. The saccharide molecules before coupling may bear substituents of interest and/or be functionalized once randomly or regioselectively coupled to one another.

Thus, by way of examples, the compounds according to the invention may be obtained according to one of the following processes:
random grafting of the substituents onto a saccharide backbone;
one or more steps of glycosylation between monosaccharide or oligosaccharide molecules bearing substituents;
one or more steps of glycosylation between one or more monosaccharide or oligosaccharide molecules bearing substituents and one or more monosaccharide or oligosaccharide molecules;
one or more steps of introducing protective groups onto alcohols or acids naturally borne by the saccharide backbone, followed by one or more substituent grafting reactions and, finally, a step of removing the protective groups;
one or more steps of glycosylation between one or more monosaccharide or oligosaccharide molecules bearing protective groups on alcohols or acids naturally borne by the saccharide backbone, one or more steps of grafting substituents onto the backbone obtained, then a step of removing the protective groups;
one or more steps of glycosylation between one or more monosaccharide or oligosaccharide molecules bearing protective groups on alcohols or acids naturally borne by the saccharide backbone, and one or more monosaccharide or oligosaccharide molecules, one or more substituent grafting steps, and then a step of removing the protective groups.

The compounds according to the invention, isolated or as a mixture, can be separated and/or purified in different ways after they have been obtained, in particular by means of the processes described above.

Mention may in particular be made of chromatography methods, in particular termed “preparative”, such as:
flash chromatography, in particular on silica, and
chromatography of the HPLC (high performance liquid chromatography) type, in particular RP-HPLH or reverse phase HPLC.

Selective precipitation methods can also be used.

The invention also relates to the use of the anionic compounds according to the invention for preparing pharmaceutical compositions.

The invention also relates to a pharmaceutical composition comprising one of the anionic compounds according to the invention as previously described and at least one active ingredient.

The invention also relates to a pharmaceutical composition characterized in that the active ingredient is chosen from the group consisting of proteins, glycoproteins, peptides and nonpeptide therapeutic molecules.

The term “active ingredient” is intended to mean a product in the form of a single chemical entity and/or in the form of a combination having a physiological activity. Said active ingredient may be exogenous, i.e. it is provided by the composition according to the invention. It may also be endogenous, for example growth factors which will be secreted into a wound during the first healing phase and which may be kept on said wound by the composition according to the invention.

Depending on the pathological conditions targeted, it is intended for local and/or systemic treatment.

In the case of local and systemic releases, the modes of administration envisioned are via the intravenous, subcutaneous, intradermal, transdermal, intramuscular, oral, nasal, vaginal, ocular, buccal, pulmonary etc. route.

The pharmaceutical compositions according to the invention are either in liquid form, in an aqueous solution, or in the form of a powder, an implant or a film. They also comprise conventional pharmaceutical excipients well known to those skilled in the art.

Depending on the pathological conditions and the modes of administration, the pharmaceutical compositions may advantageously also comprise excipients for formulating them in the form of a gel, a sponge, an injectable solution, an oral solution, an orally disintegrating tablet, etc.

The invention also relates to a pharmaceutical composition, characterized in that it is administrable in the form of a stent, a film or coating of implantable biomaterials, or an implant.

### EXAMPLES

### A. Preparation of the Compounds and Counterexamples

The structures of the compounds according to the invention are given in table 1. The structures of the counterexamples are given in table 2.

TABLE 1

| Com- | | | | Substituent | Substituent |
|---|---|---|---|---|---|
| pounds | i | j | Saccharide chain | —R′₁ | —R₁—[[Q]—[R₂]ₙ]ₘ |
| 1 | 1.0 | 0.65 | 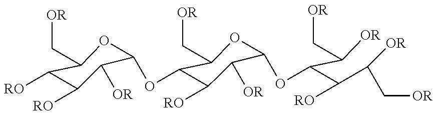 | 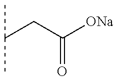 | 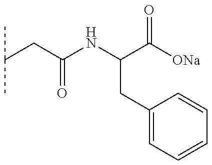 |
| 2 | 0.65 | 1.0 | 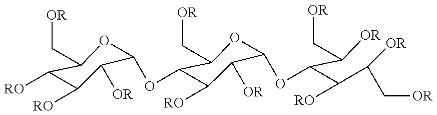 | 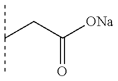 | 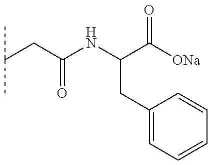 |
| 3 | 0.35 | 0.65 | 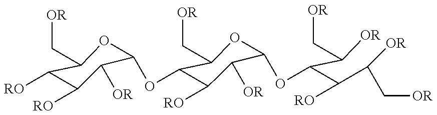 | 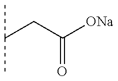 | 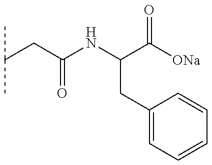 |
| 4 | 0.65 | 1.0 | 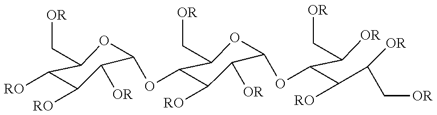 | 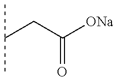 | 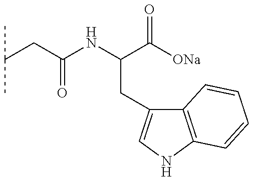 |
| 5 | 1.56 | 0.09 | 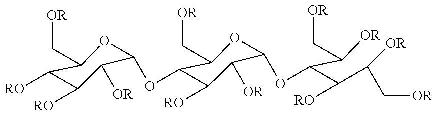 | 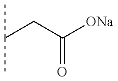 | 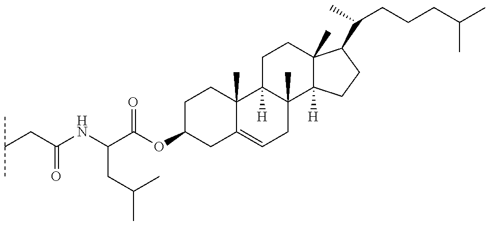 |
| 6 | 0.8 | 3.5 | 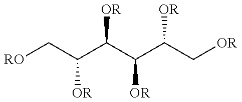 | 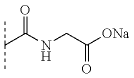 | 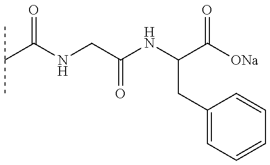 |

Compound 7: R = R₁—[[Q]—[R₂]ₙ]ₘ

| | | | | | |
|---|---|---|---|---|---|
| 7 | 0 | 6 | 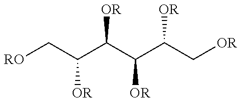 | 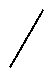 | 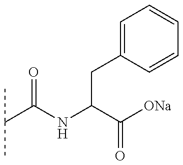 |

Compounds 8 to 30: R = H, R′₁, R₁—[[Q]—[R₂]ₙ]ₘ

| | | | | | |
|---|---|---|---|---|---|
| 8 | 1.25 | 0.4 | 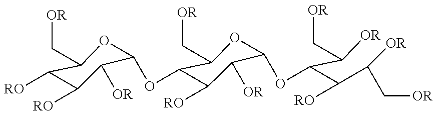 | 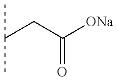 | 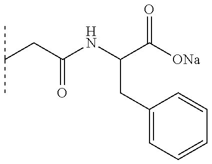 |
| 9 | 0.8 | 0.65 | 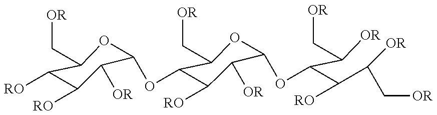 | 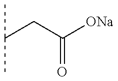 | 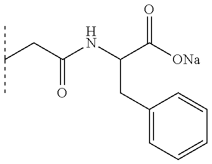 |
| 10 | 2.65 | 0.65 | 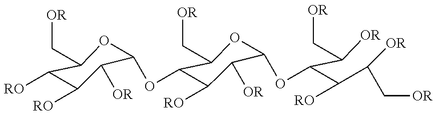 | 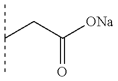 | 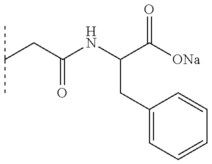 |
| 11 | 1.0 | 0.75 | 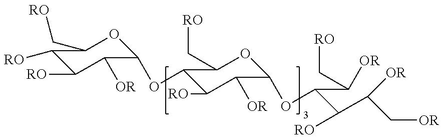 | 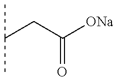 | 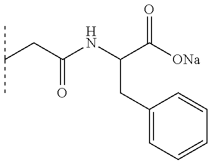 |
| 12 | 1.0 | 0.65 | 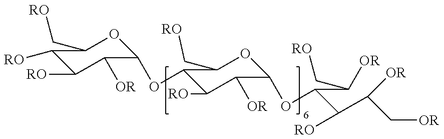 | 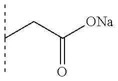 | 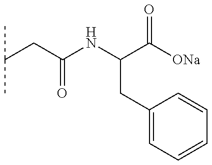 |
| 13 | 1.76 | 0.08 | 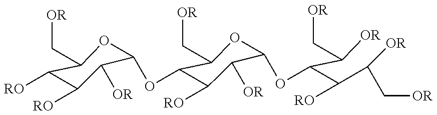 | 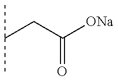 | 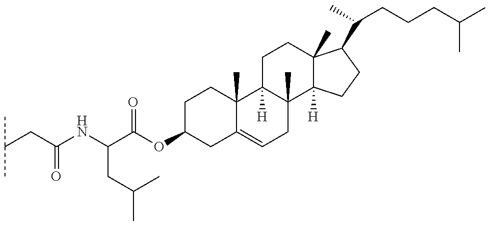 |
| 14 | 1.33 | 0.29 | 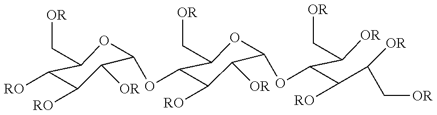 | 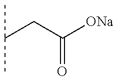 | 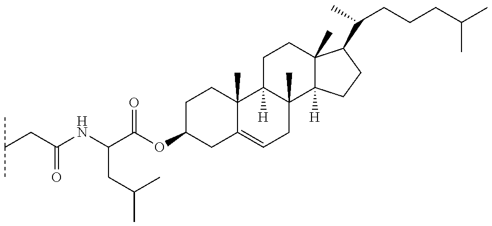 |
| 15 | 3.01 | 0.29 | 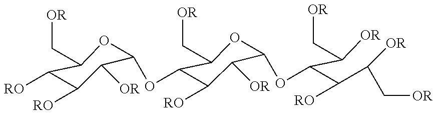 | 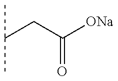 | 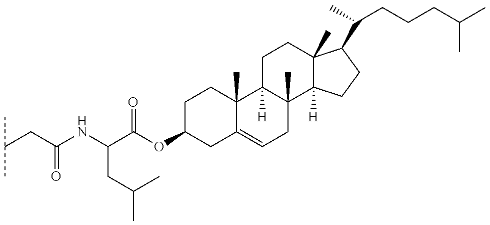 |
| 16 | 1.61 | 0.14 | 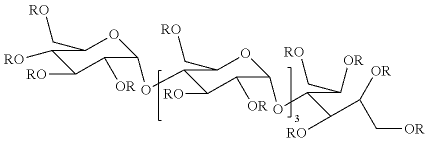 | 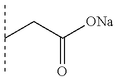 | 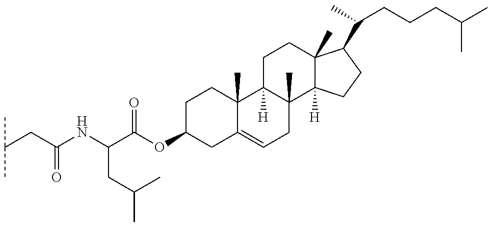 |
| 17 | 1.11 | 0.09 | 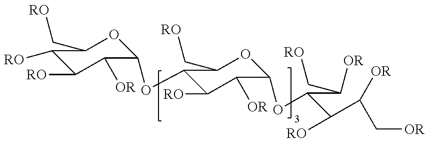 | 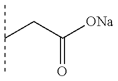 | 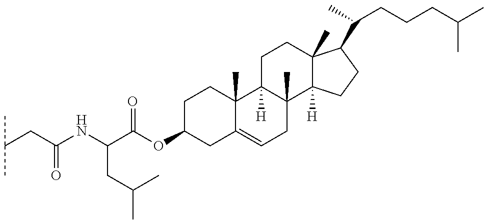 |
| 18 | 1.15 | 0.53 | 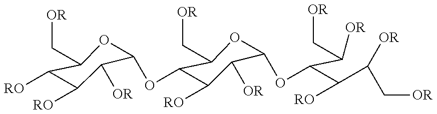 | 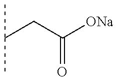 | 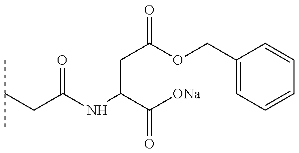 |
| 19 | 2.37 | 0.36 | 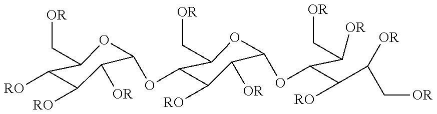 | 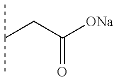 | 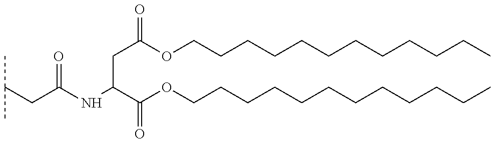 |
| 20 | 2.52 | 0.21 | 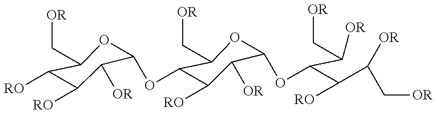 | 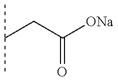 | 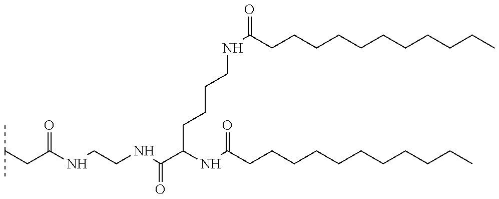 |
| 21 | 1.37 | 0.27 | 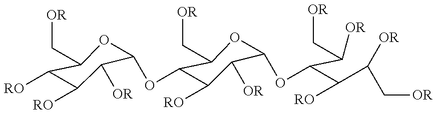 | 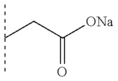 | 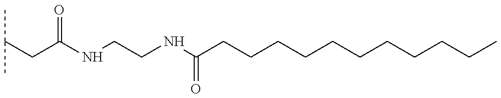 |
| 22 | 2.36 | 0.41 | 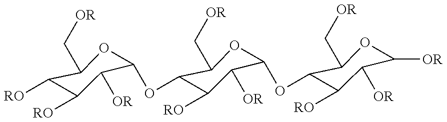 | 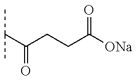 | 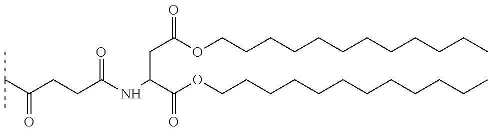 |
| 23 | 1.43 | 0.21 | 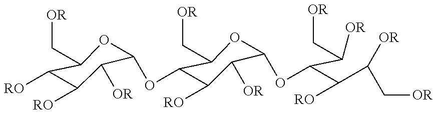 | 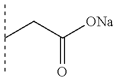 | 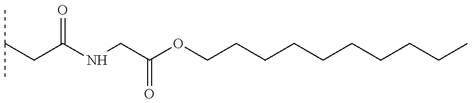 |
| 24 | 1.06 | 0.58 | 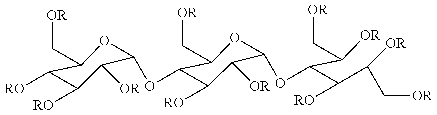 | 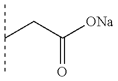 | 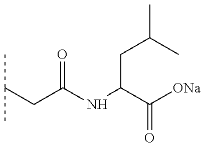 |
| 25 | 2.45 | 0.28 | 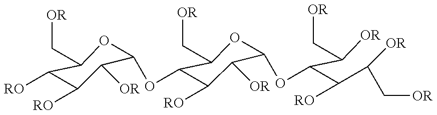 | 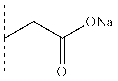 | 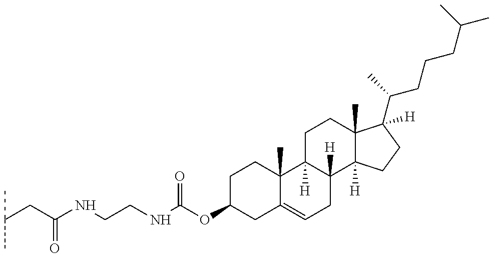 |
| 26 | 1.12 | 0.52 | 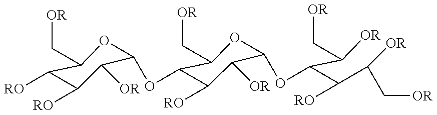 | 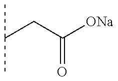 | 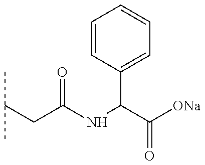 |
| 27 | 1.36 | 0.28 | 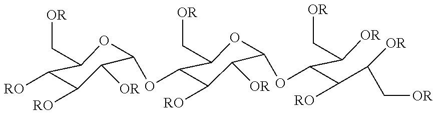 | 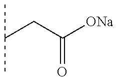 | 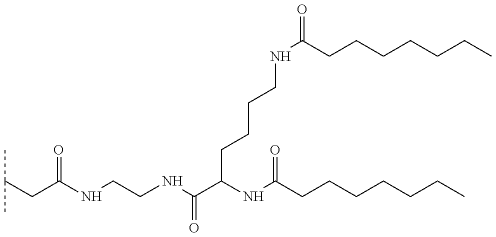 |
| 28 | 0.83 | 0.81 | 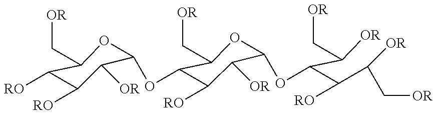 | 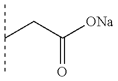 | 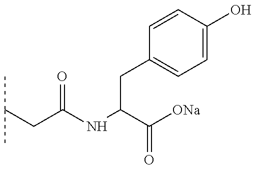 |
| 29 | 1.37 | 0.27 | 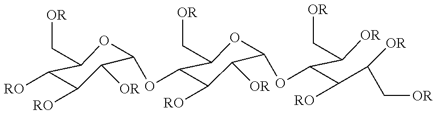 | 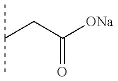 | 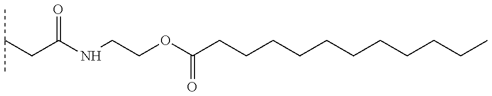 |
| 30 | 1.25 | 0.39 | 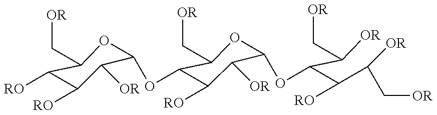 | 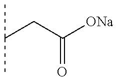 | 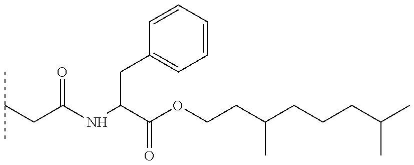 |

Compound 31: R = ONa, [Q]—[R₂]ₙ

| | | | | | |
|---|---|---|---|---|---|
| 31 | 0.28 | 0.22 | 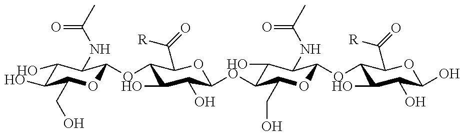 | 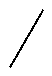 | 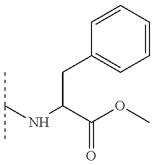 |

Compounds 32 to 33: R = H, R′₁, R₁—[[Q]—[R₂]ₙ]ₘ

| | | | | | |
|---|---|---|---|---|---|
| 32 | 1.43 | 0.21 | 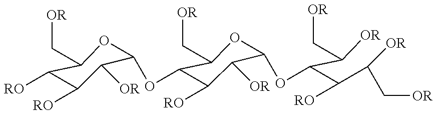 | 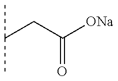 | 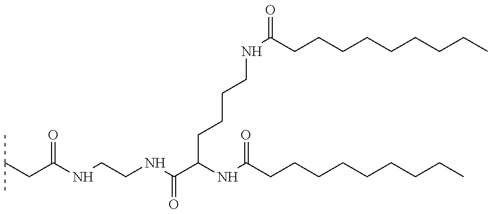 |
| 33 | 1.27 | 0.37 | 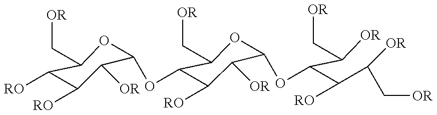 | 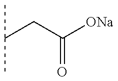 | 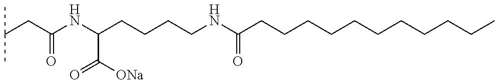 |

Compound 34: R = H, R₁—[[Q]—[R₂]ₙ]ₘ

| | | | | | |
|---|---|---|---|---|---|
| 34 | 0 | 4 | 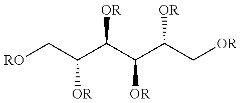 | 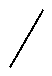 | 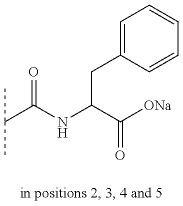 |

TABLE 2

| | | | | | | |
|---|---|---|---|---|---|---|
| | | | | Weight- | | |
| | | | | average | | |
| Coun- | | | | molar | | |
| ter | | | | mass | | |
| exam- | | | | (kg/ | Substituent | Substituent |
| ples | i | j | Saccharide chain | mol) | —R′₁ | —R₁—[[AA]—[R₂]ₙ] |

Counterexamples A1, A2, B1 and B2: R = H, R′₁, R₁—[[Q]—[R₂]ₙ]ₘ

| | | | | | | |
|---|---|---|---|---|---|---|
| A1 | 1.0 | 0.65 | 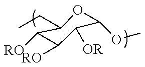 | 1 | 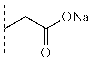 | 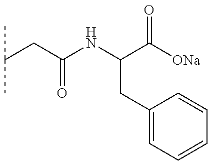 |
| A2 | 0.98 | 0.66 | 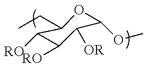 | 5 | 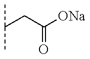 | 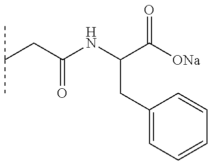 |
| B1 | 1.64 | 0.05 | 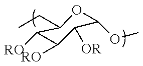 | 1 | 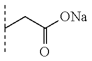 | 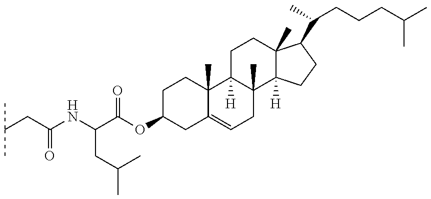 |
| B2 | 1.60 | 0.04 | 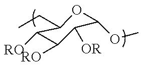 | 5 | 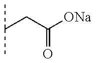 | 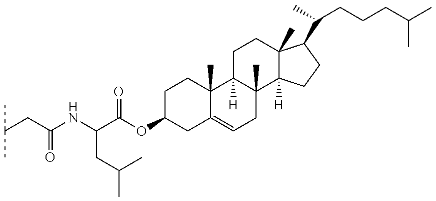 |

Compound 1: Sodium Maltotriosemethylcarboxylate Functionalized with L-Phenylalanine

0.6 g (16 mmol) of sodium borohydride is added to 8 g (143 mmol of hydroxyl functions) of maltotriose (CarboSynth) dissolved in water at 65° C. After stirring for 30 min, 28 g (238 mmol) of sodium chloroacetate are added. 24 ml of 10N NaOH (240 mmol) are then added dropwise to this solution and then the mixture is heated at 65° C. for 90 minutes. 16.6 g (143 mmol) of sodium chloroacetate are then added to the reaction medium, as are 14 ml of 10N NaOH (140 mmol), dropwise. After heating for 1 h, the mixture is diluted with water, neutralized with acetic acid and then purified by ultrafiltration on a 1 kDa PES membrane against water. The compound concentration of the final solution is determined by the dry extract, and then an acid/base assay in a 50/50 (V/V) water/acetone mixture is carried out in order to determine the degree of substitution with methylcarboxylate.

According to the dry extract: [compound]=32.9 mg/g.

According to the acid/base assay, the degree of substitution with methylcarboxylate is 1.65 per glucoside unit.

The sodium maltotriosemethylcarboxylate solution is acidified on a Purolite (anionic) resin in order to obtain maltotriosemethylcarboxylic acid which is then lyophilized for 18 hours.

10 g of maltotriosemethylcarboxylic acid (63 mmol of methylcarboxylic acid functions) are solubilized in DMF and then cooled to 0° C. A mixture of ethyl phenylalaninate, hydrochloride salt (5.7 g; 25 mmol) in DMF is prepared. 2.5 g of triethylamine (25 mmol) are added to this mixture. A solution of NMM (6.3 g; 63 mmol) and of EtOCOCl (6.8 g, 63 mmol) is then added to the mixture at 0° C. The ethyl phenylalaninate solution is then added and the mixture is stirred at 10° C. An aqueous imidazole solution (340 g/l) is added and the mixture is then heated to 30° C. The medium is diluted with water and then the solution obtained is purified by ultrafiltration on a 1 kDa PES membrane against 0.1N NaOH, 0.9% NaCl and water. The compound concentration of the final solution is determined by the dry extract. A sample of solution is lyophilized and analyzed by ¹H NMR in D₂O in order to determine the degree of substitution with methylcarboxylates functionalized with phenylalanine.

According to the dry extract: [compound 1]=28.7 mg/g

According to the ¹H NMR: the degree of substitution with methylcarboxylates functionalized with phenylalanine per glycoside unit is 0.65.

The degree of substitution with sodium methylcarboxylates per glycoside unit is 1.0.

Compound 2: Sodium Maltotriosemethylcarboxylate Functionalized with L-Phenylalanine

Using a process similar to the one used for the preparation of compound 1, a sodium maltotriosemethylcarboxylate functionalized with phenylalanine is obtained.

According to the dry extract: [compound 2]=29.4 mg/g

According to the ¹H NMR: the degree of substitution with methylcarboxylates functionalized with phenylalanine per glycoside unit is 1.0.

The degree of substitution with sodium methylcarboxylates per glycoside unit is 0.65.

Compound 3: Sodium Maltotriosemethylcarboxylate Functionalized with L-Phenylalanine

0.6 g (16 mmol) of sodium borohydride is added to 8 g (143 mmol of hydroxyl functions) of maltotriose (CarboSynth) dissolved in water at 65° C. After stirring for 30 min, 15 g (131 mmol) of sodium chloroacetate are added. 24 ml of 10N NaOH (240 mmol) are then added dropwise to this solution. After heating at 65° C. for 90 min, the mixture is diluted with water, neutralized by adding acetic acid and then purified by ultrafiltration on a 1 kDa PES membrane against water. The compound concentration of the final solution is determined by the dry extract, and then an acid/base assay in a 50/50 (V/V) water/acetone mixture is carried out in order to determine the degree of substitution with methylcarboxylate.

According to the dry extract: [compound]=20.1 mg/g.

According to the acid/base assay, the degree of substitution with methylcarboxylate is 1.0 per glycoside unit.

The sodium maltotriosemethylcarboxylate solution is acidified on a Purolite (anionic) resin in order to obtain maltotriosemethylcarboxylic acid which is then lyophilized for 18 hours.

Using a process similar to the one used for the preparation of compound 1, a sodium maltotriosemethylcarboxylate functionalized with phenylalanine is obtained.

According to the dry extract: [compound 3]=11.1 mg/g.

According to the ¹H NMR: the degree of substitution with methylcarboxylates functionalized with phenylalanine per glucoside unit is 0.65.

The degree of substitution with sodium methylcarboxylates per glucoside unit is 0.35.

Compound 4: Sodium Maltotriosemethylcarboxylate Functionalized with L-Tryptophan

Using a process similar to the one described in the preparation of compound 1, 10 g of maltotriosemethylcarboxylic acid having a degree of substitution with methylcarboxylic acid of 1.65 per glucoside unit are obtained and then lyophilized.

10 g of maltotriosemethylcarboxylic acid (63 mmol of methylcarboxylic acid functions) are solubilized in DMF and then cooled to 0° C. A solution of NMM (7.0 g; 69 mmol) and of EtOCOCl (7.5 g; 69 mmol) is then added. 11.5 g of L-tryptophan (Ajinomoto) (57 mmol) are then added and the mixture is stirred at 10° C. An aqueous imidazole solution (340 g/l) is added and the mixture is then heated to 30° C. The mixture is diluted with water and the solution obtained is purified by ultrafiltration on a 1 kDa PES membrane against 0.9% NaCl, 0.01N NaOH and water. The compound concentration of the final solution is determined by the dry extract. A sample of solution is lyophilized and analyzed by ¹H NMR in D₂O in order to determine the degree of substitution with methylcarboxylates functionalized with tryptophan.

According to the dry extract: [compound 4]=32.9 mg/g.

According to the ¹H NMR: the degree of substitution with methylcarboxylates functionalized with tryptophan per glucoside unit is 1.0.

The degree of substitution with sodium methylcarboxylates per glucoside unit is 0.65.

Compound 5: Sodium Maltotriosemethylcarboxylate Functionalized with Cholesteryl Leucinate

Using a process similar to the one described in the preparation of compound 1, 10 g of maltotriosemethylcarboxylic acid having a degree of substitution with methylcarboxylate of 1.65 per glucoside unit are obtained and then lyophilized.

Cholesteryl leucinate, para-toluenesulfonic acid salt, is prepared from cholesterol and leucine according to the process described in U.S. Pat. No. 4,826,818 (Kenji M., et al.).

10 g of maltotriosemethylcarboxylic acid (63 mmol of methylcarboxylic acid functions) are solubilized in DMF and then cooled to 0° C. A mixture of cholesteryl leucinate, para-toluenesulfonic acid salt (2.3 g; 3 mmol) in DMF is prepared. 0.4 g of triethylamine (3 mmol) is added to the mixture. Once the mixture reaches 0° C., a solution of NMM (1.9 g; 19 mmol) and of EtOCOCl (2.1 g; 19 mmol) is added. After 10 minutes, the cholesteryl leucinate solution is added and the mixture is stirred at 10° C. The mixture is then heated to 50° C. An aqueous imidazole solution (340 g/l) is added and the medium is diluted with water. The resulting solution is purified by ultrafiltration on a 1 kDa PES membrane against 0.01N NaOH, 0.9% NaCl and water. The compound concentration of the final solution is determined by the dry extract. A sample of solution is lyophilized and analyzed by ¹H NMR in D₂O in order to determine the degree of substitution with methylcarboxylates grafted with cholesteryl leucinate.

According to the dry extract: [compound 5]=10.1 mg/g.

According to the ¹H NMR: the degree of substitution with methylcarboxylates grafted with cholesteryl leucinate per glucoside unit is 0.09.

The degree of substitution with sodium methylcarboxylates per glucoside unit is 1.56.

Compound 6: Sodium N-Methylcarboxylate Mannitol Carbamate Modified with L-Phenylalanine

8 g (131 mmol of hydroxyl functions) of mannitol (Fluka) are solubilized in DMF at 80° C. After stirring for 30 minutes, DABCO (1,4-diazabicyclo[2.2.2]octane, 2.0 g; 18 mmol) and 9 ml of toluene are added to the mixture which is brought to 120° C. with stirring and hetéroazeotropically distilled. After the temperature of the reaction mixture has returned to 80° C., 34 g (263 mmol) of ethyl isocyanatoacetate are gradually introduced. After 1.5 h of reaction, the medium is precipitated from an excess of water. The solid is filtered off and saponified in an MeOH/THF mixture to which 265 ml of 1N NaOH are added at ambient temperature. The solution is stirred overnight at ambient temperature and then concentrated in a rotary evaporator. The aqueous residue is acidified on a Purolite (anionic) resin in order to obtain mannitol N-methylcarboxylic acid. The compound concentration of the final solution is determined by the dry extract, and then an acid/base assay in a 50/50 (V/V) water/acetone mixture is carried out in order to determine the degree of substitution with methylcarboxylate.

According to the dry extract: [compound]=27.4 mg/g.

According to the acid/base assay, the degree of substitution with methylcarboxylate per molecule of mannitol is 4.3.

The mannitol N-methylcarboxylic acid solution is then lyophilized for 18 hours.

10 g of mannitol N-methylcarboxylic acid (70 mmol of methylcarboxylic acid functions) are solubilized in DMF (14 g/l) and then cooled to 0° C. A mixture of ethyl phenylalaninate, hydrochloride salt (16 g; 70 mmol) in DMF is prepared (100 g/l). 7.1 g of triethylamine (70 mmol) are added to this mixture. Once the mixture reaches 0° C., a solution of NMM (7.8 g; 77 mmol) and of EtOCOCl (8.3 g; 77 mmol) is added. After 10 minutes, the ethyl phenylalaninate solution is added and the mixture is stirred at 10° C. An aqueous imidazole solution (340 g/l) is added. The solution is then heated to 30° C. and then diluted by adding water. The solution obtained is purified by ultrafiltration on a 1 kDa PES membrane against 0.1N NaOH, 0.9% NaCl and water. The compound concentration of the final solution is determined by the dry extract. A sample of solution is lyophilized and analyzed by ¹H NMR in D₂O in order to determine the degree of substitution with N-methylcarboxylates functionalized with phenylalanine.

According to the dry extract: [compound 6]=7.4 mg/g.

According to the ¹H NMR: the degree of substitution with N-methylcarboxylates functionalized with phenylalanine per molecule of mannitol is 0.35.

The degree of substitution with sodium N-methylcarboxylates per molecule of mannitol is 3.95.

### Compound 7: Sodium N-Phenylalaninate Mannitol Hexacarbamate

Ethyl L-phenylalaninate isocyanate is obtained according to the process described in the publication Tsai, J. H. et al. Organic Syntheses 2004, 10, 544-545, from ethyl L-phenylalanine hydrochloride (Bachem) and triphosgene (Sigma).

0.91 g (5 mmol) of mannitol (Fluka) is dissolved in toluene and then 8.2 g (37 mmol) of ethyl L-phenylalaninate isocyanate and 1 g (12.2 mmol) of diazabicyclo[2.2.2]octane (DABCO) are added. The mixture is heated at 90° C. overnight. After concentration under vacuum, the medium is diluted in dichloromethane and then washed with 1N HCl. The aqueous phase is extracted with dichloromethane and then the organic phases are combined, dried and concentrated under vacuum. The ethyl N-phenylalaninate mannitol hexacarbamate is isolated by flash chromatography (cyclohexane/ethyl acetate).

Yield: 4.34 g (58%).

¹H NMR (DMSO-d₆, ppm): 0.75-1.25 (6H); 2.75-3.15 (12H); 3.7-4.4 (22H); 4.8-5.2 (4H); 7.1-7.35 (30H); 7.4-7.85 (6H).

MS (ESI): 1497.7 ([M+H]⁺); ([M+H]⁺ calculated: 1498.7).

22.1 ml of 2N NaOH are added to 10.7 g (7.14 mmol) of ethyl N-phenylalaninate mannitol hexacarbamate dissolved in a tetrahydrofuran (THF)/ethanol/water mixture and the mixture is stirred at room temperature for 3 h. After evaporation of the THF and ethanol under vacuum, the residual aqueous phase is washed with dichloromethane, concentrated under vacuum and acidified with 2N HCl. The suspension is cooled to 0° C. and filtered, and then the white solid of N-phenylalanine acid mannitol hexacarbamate obtained is thoroughly washed with water and then dried under vacuum.

Yield: 9.24 g (97%).

¹H NMR (DMSO-d₆, TFA-d₁, ppm): 2.6-3.25 (12H); 3.8-4.3 (10H); 4.75-5.0 (4H); 7.0-7.75 (36H).

MS (ESI): 1329.6 ([M+H]⁺); ([M+H]⁺ calculated: 1330.4).

The N-phenylalanine acid mannitol hexacarbamate is dissolved in water (50 g/l) and neutralized by gradually adding 10N sodium hydroxide in order to give an aqueous solution of sodium N-phenylalaninate mannitol hexacarbamate which is then lyophilized.

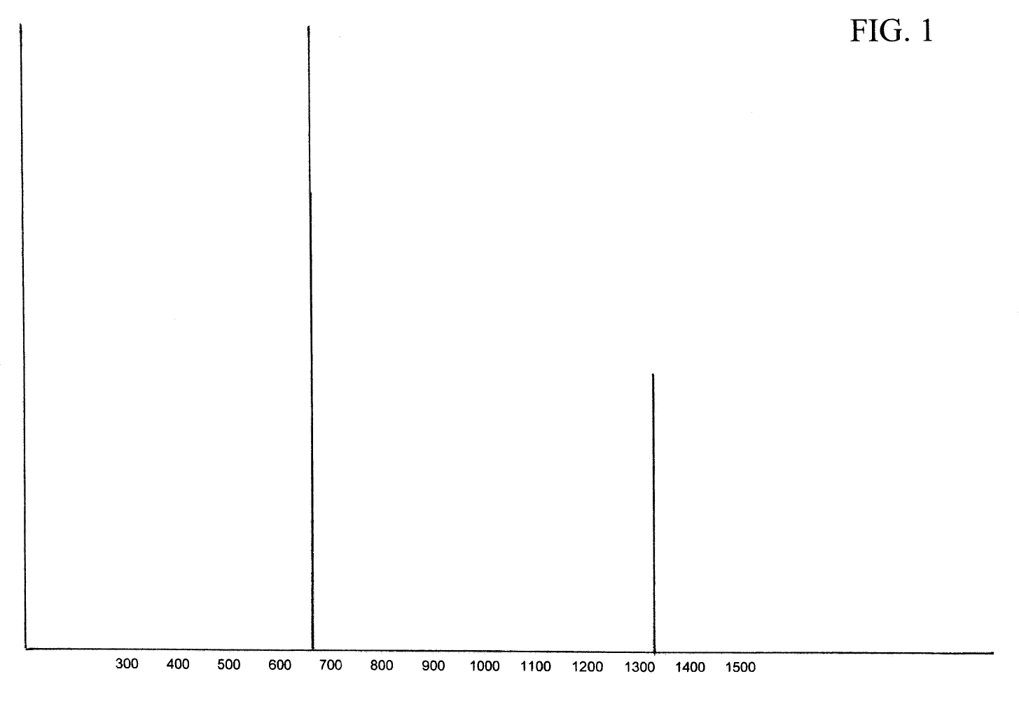

¹H NMR (D₂O, ppm): 2.6-3.25 (12H); 3.8-4.3 (10H); 4.75-5.0 (4H); 6.9-7.5 (30H). LC/MS (CH₃CN/H₂O/HCO₂H (10 mM), ELSD, ESI in negative mode): 1328.4 ([M−1]); ([M−1] calculated: 1328.3). This mass spectrum is shown in FIG. 1.

Compound 8: Sodium Maltotriosemethylcarboxylate Functionalized with L-Phenylalanine

Using a process similar to the one used for the preparation of compound 1, a sodium maltotriosemethylcarboxylate functionalized with phenylalanine is obtained.

According to the dry extract: [compound 8]=10.9 mg/g.

According to the ¹H NMR: the degree of substitution with methylcarboxylates functionalized with phenylalanine per glucoside unit is 0.40.

The degree of substitution with sodium methylcarboxylates per glucoside unit is 1.25.

Compound 9: Sodium Maltotriosemethylcarboxylate Functionalized with L-Phenylalanine

0.6 g (16 mmol) of sodium borohydride is added to 8 g (143 mmol of hydroxyl functions) of maltotriose (CarboSynth) dissolved in water at 65° C. After stirring for 30 min, 28 g (237 mmol) of sodium chloroacetate are added. 24 ml of 10N NaOH (240 mmol) are then added dropwise to this solution. After heating at 65° C. for 90 min, the mixture is diluted with water, neutralized by adding acetic acid and then purified by ultrafiltration on a 1 kDa PES membrane against water. The compound concentration of the final solution is determined by the dry extract, and then an acid/base assay in a 50/50 (V/V) water/acetone mixture is carried out in order to determine the degree of substitution with methylcarboxylate.

According to the dry extract: [compound]=14.5 mg/g.

According to the acid/base assay, the degree of substitution with methylcarboxylate is 1.45 per glucoside unit.

The sodium maltotriosemethylcarboxylate solution is acidified on a Purolite (anionic) resin in order to obtain maltotriosemethylcarboxylic acid which is then lyophilized for 18 hours.

Using a process similar to the one used for the preparation of compound 1, a sodium maltotriosemethylcarboxylate functionalized with phenylalanine is obtained.

According to the dry extract: [compound 9]=10.8 mg/g.

According to the ¹H NMR: the degree of substitution with methylcarboxylates functionalized with phenylalanine per glucoside unit is 0.65.

The degree of substitution with sodium methylcarboxylates per glucoside unit is 0.8.

Compound 10: Sodium Maltotriosemethylcarboxylate Functionalized with L-Phenylalanine

Using a process similar to the one described in the preparation of compound 1, 8 g of sodium maltotriosemethylcarboxylate characterized by a degree of substitution with sodium methylcarboxylate of 1.76 are synthesized and lyophilized.

8 g (58 mmol of hydroxyl functions) of the lyophilisate and 15 g (129 mmol) of sodium chloroacetate are dissolved in water at 65° C. 13 ml of 10N NaOH (130 mmol) are added dropwise to this solution and then the mixture is heated at 65° C. for 90 minutes. 9 g (78 mmol) of sodium chloroacetate are then added to the reaction medium, as are 8 ml of 10N NaOH (80 mmol), dropwise. After heating for 1 h, the mixture is diluted with water, neutralized with acetic acid and then purified by ultrafiltration on a 1 kDa PES membrane against water. The compound concentration of the final solution is determined by the dry extract, and then an acid/base assay in a 50/50 (V/V) water/acetone mixture is carried out in order to determine the degree of substitution with sodium methylcarboxylate

According to the dry extract: [compound]=11.7 mg/g.

According to the acid/base assay, the degree of substitution with sodium methylcarboxylate is 3.30.

The sodium maltotriosemethylcarboxylate solution is acidified on a Purolite (anionic) resin in order to obtain maltotriosemethylcarboxylic acid which is then lyophilized for 18 hours.

Using a process similar to the one used for the preparation of compound 1, a sodium maltotriosemethylcarboxylate functionalized with phenylalanine is obtained.

According to the dry extract: [compound 10]=14.9 mg/g.

According to the ¹H NMR: the degree of substitution with methylcarboxylates functionalized with phenylalanine per glucoside unit is 0.65.

The degree of substitution with sodium methylcarboxylates per glucoside unit is 2.65.

Compound 11: Sodium Maltopentaosemethylcarboxylate Functionalized with L-Phenylalanine

Using a process similar to the one described in the preparation of compound 1, but carried out with maltopentaose (CarboSynth), 10 g of maltopentaosemethylcarboxylic acid having a degree of substitution with methylcarboxylic acid of 1.75 per glucoside unit are obtained and then lyophilized.

Using a process similar to the one used for the preparation of compound 1, a sodium maltopentaosemethylcarboxylate functionalized with phenylalanine is obtained.

According to the dry extract: [compound 11]=7.1 mg/g.

According to the ¹H NMR: the degree of substitution with methylcarboxylates functionalized with phenylalanine per glucoside unit is 0.75.

The degree of substitution with sodium methylcarboxylates per glucoside unit is 1.0.

Compound 12: Sodium Maltooctaosemethylcarboxylate Functionalized with L-Phenylalanine

Using a process similar to the one described in the preparation of compound 1, but carried out with maltooctaose (CarboSynth), 10 g of maltooctaosemethylcarboxylic acid having a degree of substitution with methylcarboxylic acid of 1.65 per glucoside unit are obtained and then lyophilized.

Using a process similar to the one used for the preparation of compound 1, a sodium maltooctaosemethylcarboxylate functionalized with phenylalanine is obtained.

According to the dry extract: [compound 12]=26.3 mg/g.

According to the ¹H NMR: the degree of substitution with methylcarboxylates functionalized with phenylalanine per glucoside unit is 0.65.

The degree of substitution with sodium methylcarboxylates per glucoside unit is 1.0.

Compound 13: Sodium Maltotriosemethylcarboxylate Functionalized with Cholesteryl Leucinate

Using a process similar to the one described in the preparation of compound 5, a sodium maltotriosemethylcarboxylate, characterized by a degree of substitution with sodium methylcarboxylate of 1.84, is functionalized with cholesteryl leucinate.

According to the dry extract: [compound 13]=10.1 mg/g.

According to the ¹H NMR: the degree of substitution with methylcarboxylates functionalized with cholesteryl leucinate is 0.08.

The degree of substitution with sodium methylcarboxylates per glucoside unit is 1.76.

Compound 14: Sodium Maltotriosemethylcarboxylate Functionalized with Cholesteryl Leucinate

Using a process similar to the one described in the preparation of compound 5, a sodium maltotriosemethylcarboxylate, characterized by a degree of substitution with sodium methylcarboxylate of 1.62, is functionalized with cholesteryl leucinate.

According to the dry extract: [compound 14]=29.4 mg/g.

According to the ¹H NMR: the degree of substitution with methylcarboxylates functionalized with cholesteryl leucinate is 0.29.

The degree of substitution with sodium methylcarboxylates per glucoside unit is 1.33.

Compound 15: Sodium Maltotriosemethylcarboxylate Functionalized with Cholesteryl Leucinate

Using a process similar to the one described in the preparation of compound 10, 10 g of maltotriosemethylcarboxylic acid having a degree of substitution with methylcarboxylic acid of 3.30 per glucoside unit are obtained and then lyophilized.

Using a process similar to the one described in the preparation of compound 5, a sodium maltotriosemethylcarboxylate, characterized by a degree of substitution with sodium methylcarboxylate of 3.30, is functionalized with cholesteryl leucinate.

According to the dry extract: [compound 15]=13.1 mg/g.

According to the ¹H NMR: the degree of substitution with methylcarboxylates functionalized with cholesteryl leucinate is 0.29.

The degree of substitution with sodium methylcarboxylates per glucoside unit is 3.01.

Compound 16: Sodium Maltopentaosemethylcarboxylate Functionalized with Cholesteryl Leucinate

Using a process similar to the one described in the preparation of compound 11, 10 g of maltopentaosemethylcarboxylic acid, characterized by a degree of substitution with methylcarboxylic acid of 1.75, are synthesized and then lyophilized.

Using a process similar to the one described in the preparation of compound 5, a sodium maltopentaosemethylcarboxylate functionalized with cholesteryl leucinate is obtained.

According to the dry extract: [compound 16]=10.9 mg/g.

According to the ¹H NMR: the degree of substitution with methylcarboxylates functionalized with cholesteryl leucinate is 0.14.

The degree of substitution with sodium methylcarboxylates per glucoside unit is 1.61.

Compound 17: Sodium Maltooctaosemethylcarboxylate Functionalized with Cholesteryl Leucinate

Using a process inspired by the one described in the preparation of compound 12, 10 g of maltooctaosemethylcarboxylic acid, characterized by a degree of substitution with methylcarboxylic acid of 1.2, are synthesized and then lyophilized.

Using a process similar to the one described in the preparation of compound 5, a sodium maltooctaosemethylcarboxylate functionalized with cholesteryl leucinate is obtained.

According to the dry extract: [compound 17]=14.7 mg/g.

According to the ¹H NMR: the degree of substitution with methylcarboxylates functionalized with cholesteryl leucinate is 0.09.

The degree of substitution with sodium methylcarboxylates per glucoside unit is 1.11.

Compound 18: Sodium Maltotriosemethylcarboxylate Functionalized with β-Benzyl Aspartate

Using a process similar to the one described in the preparation of compound 1, 10 g of maltotriosemethylcarboxylic acid having a degree of substitution with methylcarboxylic acid of 1.68 per glucoside unit are obtained and then lyophilized.

6 g of maltotriosemethylcarboxylic acid (38 mmol of methylcarboxylic acid functions) are solubilized in DMF and then cooled to 0° C. A mixture of β-benzyl aspartate (Bachem, 3.5 g; 16 mmol) and of triethylamine (16 mmol) is prepared in water. A solution of NMM (3.2 g; 32 mmol) and of EtOCOCl (3.4 g, 32 mmol) is then added to the maltotriosemethylcarboxylic acid solution at 0° C. The solution of benzyl aspartate and of triethylamine is then added and the mixture is stirred at 30° C. An aqueous imidazole solution (340 g/l) is added after 90 minutes. The medium is diluted with water and then the solution obtained is purified by ultrafiltration on a 1 kDa PES membrane against a 150 mM NaHCO₃/Na₂CO₃ buffer, pH 10.4, 0.9% NaCl and water. The compound concentration of the final solution is determined by the dry extract. A sample of solution is lyophilized and analyzed by ¹H NMR in D₂O in order to determine the degree of substitution with methylcarboxylates functionalized with β-benzyl aspartate.

According to the dry extract: [compound 18]=15.0 mg/g.

According to the ¹H NMR: the degree of substitution with methylcarboxylates functionalized with β-benzyl aspartate per glucoside unit is 0.53.

The degree of substitution with sodium methylcarboxylates per glucoside unit is 1.15.

Compound 19: Sodium Maltotriosemethylcarboxylate Functionalized with Dilauryl Aspartate

Dilauryl aspartate, para-toluenesulfonic acid salt, is prepared from dodecanol and aspartic acid according to the process described in U.S. Pat. No. 4,826,818 (Kenji M., et al.).

Using a process inspired by the one described in the preparation of compound 10, 10 g of maltotriosemethylcarboxylic acid having a degree of substitution with methylcarboxylic acid of 2.73 per glucoside unit are obtained and then lyophilized.

Using a process similar to the one described in the preparation of compound 5, a sodium maltotriosemethylcarboxylate, characterized by a degree of substitution with sodium methylcarboxylate of 2.73, is functionalized with dilauryl aspartate in DMF. The medium is diluted with water and then the solution obtained is purified by dialysis on a 3.5 kDa cellulose membrane against a 150 mM NaHCO₃/Na₂CO₃ buffer, pH 10.4, 0.9% NaCl and water. The compound concentration of the final solution is determined by means of the dry extract. A sample of solution is lyophilized and analyzed by ¹H NMR in D₂O in order to determine the degree of substitution with methylcarboxylates functionalized with dilauryl aspartate.

According to the dry extract: [compound 19]=3.4 mg/g.

According to the ¹H NMR: the degree of substitution with methylcarboxylates functionalized with dilauryl aspartate is 0.36.

The degree of substitution with sodium methylcarboxylates per glucoside unit is 2.37.

Compound 20: Sodium Maltotriosemethylcarboxylate Functionalized with 2-[(2-dodecanoylamino-6-dodecanoylamino)hexanoylamino]ethanamine

The methyl ester of N,N-bis(dodecanoyl)lysine is obtained according to the process described in the publication Pal, A et al., Tetrahedron 2007, 63, 7334-7348, from the methyl ester of L-lysine, hydrochloric acid salt (Bachem) and from dodecanoic acid (Sigma). The 2-[(2-dodecanoylamino-6-dodecanoylamino)hexanoylamino]ethanamine is obtained according to the process described in U.S. Pat. No. 2,387,201 (Weiner et al.), from the methyl ester of N,N-bis(dodecanoyl)lysine and from ethylenediamine (Roth).

Using a process similar to the one described in the preparation of compound 10, 10 g of maltotriosemethylcarboxylic acid having a degree of substitution with methylcarboxylic acid of 2.73 per glucoside unit are obtained and then lyophilized.

Using a process similar to the one described in the preparation of compound 19, a sodium maltotriosemethylcarboxylate, characterized by a degree of substitution with sodium methylcarboxylate of 2.73, is functionalized with 2-[(2-dodecanoylamino-6-dodecanoylamino)hexanoylamino]ethanamine.

According to the dry extract: [compound 20]=2.4 mg/g.

According to the ¹H NMR: the degree of substitution with methylcarboxylates functionalized with 2-[(2-dodecanoylamino-6-dodecanoylamino)hexanoylamino]ethanamine is 0.21.

The degree of substitution with sodium methylcarboxylates per glucoside unit is 2.52.

Compound 21: Sodium Maltotriosemethylcarboxylate Functionalized with N-(2-aminoethyl)dodecanamide

The N-(2-aminoethyl)dodecanamide is obtained according to the process described in U.S. Pat. No. 2,387,201 (Weiner et al.), from the methyl ester of dodecanoic acid (Sigma) and from ethylenediamine (Roth).

Using a process similar to the one described in the preparation of compound 10 g of maltotriosemethylcarboxylic acid having a degree of substitution with methylcarboxylic acid of 1.64 per glucoside unit are obtained and then lyophilized.

Using a process similar to the one described in the preparation of compound 19, a sodium maltotriosemethylcarboxylate, characterized by a degree of substitution with sodium methylcarboxylate of 1.64, is functionalized with N-(2-aminoethyl)dodecanamide.

According to the dry extract: [compound 21]=2.4 mg/g.

According to the ¹H NMR: the degree of substitution with methylcarboxylates functionalized with N-(2-aminoethyl)dodecanamide is 0.27.

The degree of substitution with sodium methylcarboxylates per glucoside unit is 1.37.

Compound 22: Sodium Maltotriosesuccinate Functionalized with Dilauryl Aspartate

25 g (i.e. 0.543 mol of hydroxyl functions) of maltotriose are solubilized in 62 ml of DMSO at 60° C., and then the temperature is programmed at 40° C. 59.3 g (0.592 mmol) of succinic anhydride in solution in 62 ml of DMF and 59.9 g (0.592 mmol) of NMM, diluted in 62 ml of DMF, are added to this solution. After 3 h of reaction, the reaction medium is diluted in water (67 ml) and the oligosaccharide is purified by ultrafiltration. The molar fraction of succinic ester formed per glucoside unit is 2.77 according to the ¹H NMR in D₂O/NaOD.

The sodium maltotriosesuccinate solution is acidified on a Purolite (anionic) resin in order to obtain maltotriosesuccinic acid which is then lyophilized for 18 hours.

Using a process similar to the one described in the preparation of compound 19, a sodium maltotriosesuccinate, characterized by a degree of substitution with sodium succinate of 2.77, is functionalized with dilauryl aspartate.

According to the dry extract: [compound 22]=12.9 mg/g.

According to the ¹H NMR: the degree of substitution with succinates functionalized with dilauryl aspartate is 0.41.

The degree of substitution with sodium methylcarboxylates per glucoside unit is 2.36.

Compound 23: Sodium Maltotriosemethylcarboxylate Functionalized with Decanoyl Glycinate

The decanoyl glycinate, para-toluenesulfonic acid salt, is prepared from decanol and from glycine according to the process described in U.S. Pat. No. 4,826,818 (Kenji M., et al.).

Using a process similar to the one described in the preparation of compound 21, a sodium maltotriosemethylcarboxylate, characterized by a degree of substitution with sodium methylcarboxylate of 1.64, is functionalized with decanoyl glycinate.

According to the dry extract: [compound 23]=2.4 mg/g.

According to the ¹H NMR: the degree of substitution with methylcarboxylates functionalized with decanoyl glycinate is 0.21.

The degree of substitution with sodium methylcarboxylates per glucoside unit is 1.43.

Compound 24: Sodium Maltotriosemethylcarboxylate Functionalized with L-Leucine

Using a process similar to the one described in the preparation of compound 18, but involving L-leucine (Roth), a sodium maltotriosemethylcarboxylate, characterized by a degree of substitution with sodium methylcarboxylate of 1.64, is functionalized with L-leucine.

According to the dry extract: [compound 24]=2.3 mg/g.

According to the ¹H NMR: the degree of substitution with methylcarboxylates functionalized with L-leucine is 0.58.

The degree of substitution with sodium methylcarboxylates per glucoside unit is 1.06.

Compound 25: Sodium Maltotriosemethylcarboxylate Functionalized with Cholesteryl 2-Aminoethylcarbamate

The cholesteryl 2-aminoethylcarbamate, hydrochloric acid salt, is prepared according to the process as described in patent WO 2010/053140 (Akiyoshi, K et al.).

Using a process similar to the one described in the preparation of compound 19, a sodium maltotriosemethylcarboxylate, characterized by a degree of substitution with sodium methylcarboxylate of 2.73, is functionalized with cholesteryl 2-aminoethylcarbamate.

According to the dry extract: [compound 25]=2.9 mg/g.

According to the ¹H NMR: the degree of substitution with methylcarboxylates functionalized with cholesteryl 2-aminoethylcarbamate is 0.28.

The degree of substitution with sodium methylcarboxylates per glucoside unit is 2.45.

Compound 26: Sodium Maltotriosemethylcarboxylate Functionalized with Alpha-Phenylglycine

Using a process similar to the one described in the preparation of compound 18, but involving alpha-phenylglycine (Bachem), a sodium maltotriosemethylcarboxylate, characterized by a degree of substitution with sodium methylcarboxylate of 1.64, is functionalized with alpha-phenylglycine.

According to the dry extract: [compound 26]=9.1 mg/g.

According to the ¹H NMR: the degree of substitution with methylcarboxylates functionalized with alpha-phenylglycine is 0.52.

The degree of substitution with sodium methylcarboxylates per glucoside unit is 1.12.

Compound 27: Sodium Maltotriosemethylcarboxylate Functionalized with 2-[(2-octanoylamino-6-octanoylamino)hexanoylamino]ethanamine

The methyl ester of N,N-bis(octanoyl)lysine is obtained according to the process described in the publication Pal, A et al., Tetrahedron 2007, 63, 7334-7348, from the methyl ester of L-lysine, hydrochloric acid salt (Bachem) and from octanoic acid (Sigma). The 2-[(2-octanoylamino-6-octanoylamino)hexanoylamino]ethanamine is obtained according to the process described in U.S. Pat. No. 2,387,201 (Weiner et al.), from the methyl ester of N,N-bis(octanoyl)lysine and from ethylenediamine (Roth).

Using a process similar to the one described in the preparation of compound 21, a sodium maltotriosemethylcarboxylate, characterized by a degree of substitution with sodium methylcarboxylate of 1.64, is functionalized with 2-[(2-octanoylamino-6-octanoylamino)hexanoylamino]ethanamine.

According to the dry extract: [compound 27]=3.8 mg/g.

According to the ¹H NMR: the degree of substitution with methylcarboxylates functionalized with 2-[(2-octanoylamino-6-octanoylamino)hexanoylamino]ethanamine is 0.28.

The degree of substitution with sodium methylcarboxylates per glucoside unit is 1.36.

Compound 28: Sodium Maltotriosemethylcarboxylate Functionalized with L-Tyrosine

Using a process similar to the one described in the preparation of compound 1, but involving methyl tyrosinate, hydrochloric acid salt (Bachem), a sodium maltotriosemethylcarboxylate, characterized by a degree of substitution with sodium methylcarboxylate of 1.64, is functionalized with tyrosine.

According to the dry extract: [compound 28]=9.1 mg/g.

According to the ¹H NMR: the degree of substitution with methylcarboxylates functionalized with L-tyrosine is 0.81.

The degree of substitution with sodium methylcarboxylates per glucoside unit is 0.83.

Compound 29: Sodium Maltotriosemethylcarboxylate Functionalized with 2-Aminoethyl Dodecanoate

The 2-aminoethyl dodecanoate, para-toluenesulfonic acid salt, is obtained according to the process described in U.S. Pat. No. 4,826,818 (Kenji M et al.), from dodecanoic acid (Sigma) and from ethanolamine (Sigma).

Using a process similar to the one described in the preparation of compound 21, a sodium maltotriosemethylcarboxylate, characterized by a degree of substitution with sodium methylcarboxylate of 1.64, is functionalized with 2-aminoethyl dodecanoate.

According to the dry extract: [compound 29]=1.8 mg/g.

According to the ¹H NMR: the degree of substitution with methylcarboxylates functionalized with 2-aminoethyl dodecanoate is 0.27.

The degree of substitution with sodium methylcarboxylates per glucoside unit is 1.37.

Compound 30: Sodium Maltotriosemethylcarboxylate Functionalized with 3,7-Dimethyloctanoyl Phenylalaninate

The 3,7-dimethyloctanoyl phenylalaninate, para-toluenesulfonic acid salt, is prepared from 3,7-dimethyloctan-1-ol and from L-phenylalanine according to the process described in U.S. Pat. No. 4,826,818 (Kenji et al.).

Using a process similar to the one described in the preparation of compound 21, a sodium maltotriosemethylcarboxylate, characterized by a degree of substitution with sodium methylcarboxylate of 1.64, is functionalized with 3,7-dimethyloctanoyl phenylalaninate.

According to the dry extract: [compound 30]=3.3 mg/g.

According to the ¹H NMR: the degree of substitution with methylcarboxylates functionalized with 3,7-dimethyloctanoyl phenylalaninate is 0.39.

The degree of substitution with sodium methylcarboxylates per glucoside unit is 1.25.

Compound 31: Sodium Hyaluronate Tetrasaccharide Functionalized with Methyl Phenylalaninate

A solution of 4-mer sodium hyaluronate (Contipro Biotech) at 30 g/l is acidified on a Purolite (anionic) resin in order to obtain an aqueous hyaluronic acid solution of which the pH is brought to 7.1 by adding an aqueous solution (40%) of tetrabutylammonium hydroxide (Sigma). The solution is then lyophilized for 18 hours.

30 mg of tetrabutylammonium hyaluronate (48 μmol of tetrabutylammonium carboxylate functions) are solubilized in DMF. 5 mg of methyl phenylalaninate (24 μmol), 6 mg of triethylamine (60 μmol) and 9 mg of 2-chloro-1-methylpyridinium iodide (Sigma, 36 μmol) are added at 0° C. and the medium is then stirred at 20° C. for 16 hours. The solution is evaporated and the residue is analyzed by ¹H NMR in D₂O in order to determine the degree of acid functions functionalized with methyl phenylalaninate.

According to the ¹H NMR: the degree of substitution with carboxylates functionalized with methyl phenylalaninate per saccharide unit is 0.22.

The degree of substitution with sodium carboxylates per saccharide unit is 0.28.

Compound 32: Sodium Maltotriosemethylcarboxylate Functionalized with 2-[(2-decanoylamino-6-decanoylamino)hexanoylamino]ethanamine

The methyl ester of N,N-bis(decanoyl)lysine is obtained according to the process described in the publication Pal, A et al., Tetrahedron 2007, 63, 7334-7348, from the methyl ester of L-lysine, hydrochloric acid salt (Bachem) and from decanoic acid (Sigma). The 2-[(2-decanoylamino-6-decanoylamino)hexanoylamino]ethanamine is obtained according to the process described in U.S. Pat. No. 2,387,201 (Weiner et al.), from the methyl ester of N,N-bis(decanoyl)lysine and from ethylenediamine (Roth).

Using a process similar to the one described in the preparation of compound 21, a sodium maltotriosemethylcarboxylate, characterized by a degree of substitution with sodium methylcarboxylate of 1.64, is functionalized with 2-[(2-decanoylamino-6-decanoylamino)hexanoylamino]ethanamine.

According to the dry extract: [compound 32]=3.9 mg/g.

According to the ¹H NMR: the degree of substitution with methylcarboxylates functionalized with 2-[(2-decanoylamino-6-decanoylamino)hexanoylamino]ethanamine is 0.21.

The degree of substitution with sodium methylcarboxylates per glucoside unit is 1.43.

Compound 33: Sodium Maltotriosemethylcarboxylate Functionalized with ε-N-dodecanoyl-L-lysine

The ethyl ester of ε-N-dodecanoyl-L-lysine, hydrochloric acid salt, is prepared from dodecanoic acid (Sigma) and from the ethyl ester of L-lysine, hydrochloric acid salt (Bachem), according to the process described in U.S. Pat. No. 4,126,628 (Paquet A M).

Using a process similar to the one described in the preparation of compound 1, a sodium maltotriosemethylcarboxylate, characterized by a degree of substitution with sodium methylcarboxylate of 1.64, is functionalized with ε-N-dodecanoyl-L-lysine.

According to the dry extract: [compound 33]=4.2 mg/g.

According to the ¹H NMR: the degree of substitution with methylcarboxylates functionalized with ε-N-dodecanoyl-L-lysine is 0.37.

The degree of substitution with sodium methylcarboxylates per glucoside unit is 1.27.

Compound 34: Sodium N-Phenylalaninate Mannitol 2,3,4,5-Tetracarbamate

1,6-ditriisopropylsilyl mannitol is obtained according to the process described in the publication Bhaskar, V et al., Journal of Carbohydrate Chemistry 2003, 22(9), 867-879.

Using a process similar to the one described for the preparation of compound 7, [1,6-ditriisopropylsilyl-2,3,4,5-tetra(sodium N-phenylalaninate carbamate)]mannitol is obtained.

Using a process similar to the one described in the publication P J Edwards et al., Synthesis 1995, 9, 898-900, the triisopropylsilyl groups are deprotected in order to give N-phenylalanine acid mannitol 2,3,4,5-tetracarbamate.

Using a process similar to the one described for the preparation of compound 7, sodium N-phenylalaninate mannitol 2,3,4,5-tetracarbamate is then obtained.

¹H NMR (D₂O, ppm): 2.6-3.25 (8H); 3.6-4.3 (8H); 4.75-5.0 (4H); 6.9-7.5 (24H).

Counterexample A1: Sodium Dextranmethylcarboxylate Functionalized with L-Phenylalanine

A sodium dextranmethylcarboxylate functionalized with L-phenylalanine is synthesized from a dextran having a weight-average molar mass of 1 kg/mol (Pharmacosmos, average degree of polymerization of 3.9) according to a process similar to the one described in application WO 2012/153070.

The degree of substitution with sodium methylcarboxylates per glucoside unit is 1.0.

The degree of substitution with methylcarboxylates functionalized with L-phenylalanine per glucoside unit is 0.65.

Counterexample A2: Sodium Dextranmethylcarboxylate Functionalized with L-Phenylalanine

A sodium dextranmethylcarboxylate functionalized with L-phenylalanine is synthesized from a dextran having a weight-average molar mass of 5 kg/mol (Pharmacosmos, average degree of polymerization of 19) according to a process similar to the one described in application WO 2010/122385.

The degree of substitution with sodium methylcarboxylates per glucoside unit is 0.98.

The degree of substitution with methylcarboxylates functionalized with L-phenylalanine per glucoside unit is 0.66.

Counterexample B1: Sodium Dextranmethylcarboxylate Functionalized with Cholesteryl Leucinate

A sodium dextranmethylcarboxylate functionalized with cholesteryl leucinate is synthesized from a dextran having a weight-average molar mass of 1 kg/mol (Pharmacosmos, average degree of polymerization of 3.9) according to a process similar to the one described in application WO 2012/153070.

The degree of substitution with sodium methylcarboxylates per glucoside unit is 1.64.

The degree of substitution with methylcarboxylates functionalized with cholesteryl leucinate per glucoside unit is 0.05.

Counterexample B2: Sodium Dextranmethylcarboxylate Functionalized with Cholesteryl Leucinate

A sodium dextranmethylcarboxylate functionalized with cholesteryl leucinate is synthesized from a dextran having a weight-average molar mass of 5 kg/mol (Pharmacosmos, average degree of polymerization of 19) according to a process similar to the one described in application WO 2010/041119.

The degree of substitution with sodium methylcarboxylates per glucoside unit is 1.60.

The degree of substitution with methylcarboxylates functionalized with cholesteryl leucinate per glucoside unit is 0.04.

### B. Turbidity Measurement Assays

The turbidity of solutions in which a “model” protein, lysozyme and either a compound according to the invention or a counterexample compound are brought together is analyzed in the compound/lysozyme molar ratios of 0, 0.1 and 0.5.

The following solutions are prepared beforehand: histidine buffer solution, pH 6.2±0.1, at 194 mM (30 mg/ml), sodium chloride (NaCl) solution at 5017 mM (293 mg/ml), solution of lysozyme (Sigma-Aldrich, Ref L6876, CAS #12650-88-3) at 15 mg/ml (0.35 mM), and solutions of each of the test products (pH 6.2±0.1), i.e. compounds according to the invention and counterexamples.

For each of the solutions of compounds to be prepared, 3 ml of an aqueous solution of compound are adjusted to pH 6.2±0.1 using 50±25 μl of a 0.1N hydrochloric acid (HCl) solution.

The solutions of the compounds tested are detailed in the following table 3.

TABLE 3

| | Final concentration of the | pH |
|---|---|---|
| Products tested | compounds (mM) | of the final solutions |
| Compound 1 | 6.8 | 6.3 |
| Counterexample A1 | 27.2 | 6.2 |
| Counterexample A2 | 5.4 | 6.3 |
| Compound 13 | 9.6 | 6.3 |
| Counterexample B1 | 10.7 | 6.2 |
| Counterexample B2 | 5.3 | 6.3 |

The test solutions at the compound/lysozyme molar ratios: 0, 0.1 and 0.5 are then prepared as follows.

The sodium chloride (NaCl) solution at 5017 mM, the histidine buffer solution at 194 mM and then the solution of compound are successively added to water, which results in a mixture which is homogenized on a roller mixer (Stuart Roller Mixer SRT9D) for 1 minute.

The lysozyme solution is, finally, added and then the final mixture is homogenized on the roller mixer for 1 minute.

The turbidity (expressed in NTU) for each of the final test solutions is measured using a HACH 2100AN turbidity meter.

The turbidity of the compound 1/lysozyme solution is analyzed in comparison with that of the counterexample A1/lysozyme and counterexample A2/lysozyme solutions. The turbidity of the compound 13/lysozyme solution is analyzed in comparison with that of the counterexample B1/lysozyme and counterexample B2/lysozyme solutions. The results are shown in the following table 4.

TABLE 4

| | Turbidity of the | Turbidity of the | Turbidity of the |
|---|---|---|---|
| | solutions at the | solutions at the | solutions at the |
| | molar ratio 0 | molar ratio 0.1 | molar ratio 0.5 |
| | (NTU) | (NTU) | (NTU) |
| Compound 1- | I | 55 | 4.8 |
| lysozyme solution | 0 | | |
| Counterexample A1- | 0 | 161 | 2480 |
| lysozyme solution | | | |
| Counterexample A2- | 0 | 1293 | 9386 |
| lysozyme solution | | | |
| Compound 13- | 0 | 32 | 395 |
| lysozyme solution | | | |
| Counterexample B1- | 0 | 90 | 768 |
| lysozyme solution | | | |
| Counterexample B2- | 0 | 1824 | Saturation |
| lysozyme solution | | | |

The turbidity of the compound 1/lysozyme solution is lower than that of the counterexample compound A1/lysozyme and counterexample compound A2/lysozyme solutions, whatever the ratio.

The turbidity of the compound 13/lysozyme solution is lower than that of the counterexample compound B1/lysozyme and counterexample compound B2/lysozyme solutions, whatever the ratio.

### C. Interaction with Albumin

It is known that the prior art compounds which do not make it possible to obtain nonturbid solutions with lysozyme, interact with proteins, in particular with “model” proteins such as albumin.

In order to determine, following the results obtained with the compounds according to the invention in the test with lysozyme (i.e. turbidity measurement assays previously described), whether there are nevertheless “model” proteins with which the compounds according to the invention may interact, a test for interaction with albumin was carried out.

The test carried out is a “fluorescence” test with albumin, which makes it possible, by measuring the variations in fluorescence of albumin, to verify whether an interaction exists between the compound tested and albumin.

The compound/albumin solutions are prepared from stock solutions of compounds and of serum albumin (BSA) by mixing the appropriate volumes in order to obtain a fixed BSA concentration at 0.5 mg/ml and compound/BSA weight ratios of 1, 5 and 10. These solutions are prepared in a PBS buffer at pH 7.4.

200 μl of the various compound/BSA solutions are introduced into a 96-well plate. The fluorescence measurements are carried out at room temperature (20° C.) with an EnVision® fluorescence spectrometer (PerkinElmer). The excitation wavelength is 280 nm and the emission wavelength is 350 nm. This corresponds to the fluorescence of the tryptophan residues of albumin (Ruiz-P. et al., M, A. Physico-chemical studies of molecular interactions between non-ionic surfactants and bovine serum albumin, Colloids Surf. B Biointerfaces 2009). The F (compound/BSA)/F0 (BSA alone) ratio makes it possible to evaluate the interaction between the compound and albumin. If this ratio is less than 1, this means that the compound induces partial quenching of the albumin fluorescence linked to a change in environment of the tryptophan residues. This change reflects an interaction between the compound and albumin. It was verified, as a control, that, for all the compounds tested, the fluorescence of the compound alone is negligible considering the fluorescence of albumin (fluorescence (compound)<2% fluorescence (albumin)). The results are given in table 5.

TABLE 5

| | Compound/BSA | Result | Result |
|---|---|---|---|
| Compound | weight ratio | F/F0 < 0.5 | F/F0 < 0.85 |
| 19 | 1 | YES | — |
| 20 | 1 | YES | — |
| 21 | 1 | YES | — |
| 22 | 1 | YES | — |
| 23 | 1 | YES | — |
| 27 | 1 | YES | — |
| 29 | 1 | YES | — |
| 30 | 1 | YES | — |
| 2 | 1 | NO | NO |
| | 5 | NO | YES |
| | 10 | NO | YES |

The results show that all the compounds interact with albumin.

As regards compounds 19 to 30, they cause a decrease in the fluorescence ratio such that F/F0<0.5 at a compound/BSA weight ratio of 1.

As regards compound 2, it decreases the fluorescence ratio such that F/F0<0.85 at a compound/BSA weight ratio of 5 and of 10.

1. Substituted anionic compounds, in isolated form or as a mixture, consisting of a backbone made up of a discrete number u of between 1 and 8 (1≦u≦8) of identical or different saccharide units, linked via identical or different glycosidic bonds, said saccharide units being chosen from the group consisting of pentoses, hexoses, uronic acids, N-acetylhexosamines in cyclic form or in open reduced form, wherein they are substituted with:
a) at least one substituent of general formula I:
—[R₁]ₐ—[[Q]-[R₂]ₙ]ₘ Formula I
the substituents being identical or different when there are at least two substituents, in which:
if n is equal to 0, then the radical -[Q]- is derived from a C₃ to C₁₅ carbon-based chain which is optionally branched or substituted, optionally unsaturated and/or optionally comprising one or more ring(s) and/or comprising at least one heteroatom chosen from O, N and S and at least one function L chosen from amine and alcohol functions, said radicals -[Q]- being attached to the backbone of the compound by means of a linker arm R₁ to which it is bonded via a function T, or directly bonded to the backbone via a function G,
if n is equal to 1 or 2, then the radical -[Q]- is derived from a C₂ to C₁₅ carbon-based chain which is optionally branched or substituted, optionally unsaturated and/or optionally comprising one or more ring(s) and/or comprising at least one heteroatom chosen from O, N and S and at least one function L chosen from amine and alcohol functions and bearing n radical(s) R₂, said radical -[Q]- being attached to the backbone of the compound by means of a linker arm R₁ to which it is bonded via a function T, or directly bonded to the backbone via a function G,
the radical —R₁— being:
either a bond and then a=0, and the radical -[Q]- is directly bonded to the backbone via a function G,
or a C₂ to C₁₅ carbon-based chain, and then a=1, which is optionally substituted and/or comprising at least one heteroatom chosen from O, N and S and at least one acid function before the reaction with the radical -[Q]-, said chain being bonded to the radical -[Q]- via a function T resulting from the reaction of the acid function of the radical —R₁— with an alcohol or amine function of the radical -[Q]-, and said radical R₁ is attached to the backbone by means of a function F resulting from a reaction between a hydroxyl function or a carboxylic acid function borne by the backbone, and the precursor of the radical —R₁—,
the radical —R₂ is a C₁ to C₃₀ carbon-based chain which is optionally branched or substituted, optionally unsaturated and/or optionally comprising on or more ring(s) and/or one or more heteroatom(s) chosen from O, N and S; it forms, with the radical -[Q]-, a function Z resulting from a reaction between the alcohol, amine or acid functions borne by the precursors of the radical —R₂ and of the radical -[Q]-,
F is a function chosen from ether, ester, amide or carbamate functions,
T is a function chosen from amide or ester functions,
Z is a function chosen from ester, carbamate, amide or ether functions,
G is a function chosen from ester, amide or carbamate functions,
n is equal to 0, 1 or 2,
m is equal to 1 or 2,
the degree of substitution of the saccharide units, j, with —[R₁]ₐ-[[AA]-[R₂]ₙ]ₘ being between 0.01 and 6, 0.01≦j≦6;
b) and, optionally, one or more substituents —R′₁,
the substituent —R′₁ being a C₂ to C₁₅ carbon-based chain which is optionally substituted and/or comprising at least one heteroatom chosen from O, N and S and at least one acid function in the form of an alkaline metal cation salt, said chain being bonded to the backbone via a function F′ resulting from a reaction between a hydroxyl function or a carboxylic acid function borne by the backbone, and the precursor of the substituent —R′₁,
the degree of substitution of the saccharide units, i, with —R′₁, being between 0 and 6-j, 0≦i≦6-j, and
if n≠0 and if the backbone does not bear anionic charges before substitution, then i≠0,
—R′₁ identical to or different than —R₁—,
the free salifiable acid functions borne by R′₁ are in the form of alkaline metal cation salts,
F′ is a function chosen from ether, ester, amide or carbamate functions,
F, F′, T, Z and G being identical or different,
i+j≦6.

2. The compounds as claimed in claim 1, wherein the radical -[Q]- is derived from an alpha-amino acid.

3. The compounds as claimed in claim 1, wherein the radical -[Q]- is chosen from diamines.

4. The compounds as claimed in claim 1, wherein the radical -[Q]- is chosen from amino alcohols.

5. The compounds as claimed in claim 1, wherein the radical -[Q]- is chosen from dialcohols.

6. The compounds as claimed in claim 2, which are substituted with:
a) at least one substituent of general formula II:
—[R₁]ₐ-[[AA]-[R₂]ₙ]ₘ Formula II
the substituents being identical or different when there are at least two substituents, in which:
if n is equal to 0, then the radical -[AA]- denotes an amino acid residue comprising a C3 to C15 carbon-based chain directly bonded to the backbone via a function G′,
if n is equal to 1 or 2, then the radical -[AA]- denotes an amino acid residue comprising a C2 to C15 carbon-based chain bearing n radical(s) —R2 attached to the backbone of the compound by means of a linker arm R1 to which it is bonded via an amide function, or directly bonded to the backbone via a function G′,
the radical —R1- being:
either a bond and then a=0, and the amino acid residue -[AA]- is directly bonded to the backbone via a function G′,
or a C2 to C15 carbon-based chain, and then a=1, which is optionally substituted and/or comprising at least one heteroatom chosen from O, N and S and at least one acid function before the reaction with the amino acid, said chain forming, with the amino acid residue -[AA]-, an amide function, and is attached to the backbone by means of a function F resulting from a reaction between a hydroxyl function or carboxylic acid function borne by the backbone, and the precursor of the radical —R₁—,
the radical —R2 is a C1 to C30 carbon-based chain which is optionally branched or substituted, optionally unsaturated and/or optionally comprising one or more ring(s) and/or one or more heteroatom(s) chosen from O, N and S; it forms, with the amino acid residue -[AA]-, a function Z′ resulting from a reaction between a hydroxyl, acid or amine function borne by the precursor of the radical —R2 and an acid function borne by the precursor of the radical -[AA]-,
F is a function chosen from ether, ester, amide or carbamate functions,
G′ is a function chosen from ester, amide or carbamate functions,
Z′ is a function chosen from ester, amide or carbamate functions,
n is equal to 0, 1 or 2,
m is equal to 1 or 2,
the degree of substitution of the saccharide units, j, with —[R1]ₐ-[[AA]-[R2]n]m being between 0.01 and 6, 0.01≦j≦6;
b) and, optionally, one or more substituents —R′₁,
the substituent —R′₁ being a C₂ to C₁₅ carbon-based chain which is optionally substituted and/or comprising at least one heteroatom chosen from O, N and S and at least one acid function in the form of an alkali metal cation salt, said chain being bonded to the backbone via a function F′ resulting from a reaction between a hydroxyl function or a carboxylic acid function borne by the backbone, and the precursor of the substituent —R′₁,
the degree of substitution of the saccharide units, i, with —R′₁, being between 0 and 6-j, 0≦i≦6-j, and
if n≠0 and if the backbone does not bear anionic charges before substitution, then i≠0,
—R′₁ identical to or different than —R₁—,
the free salifiable acid functions borne by the substituent —R′₁ are in the form of alkali metal cation salts,
F′ is an ether, ester, amide or carbamate function,
F, F′, G′ and Z′ are identical or different,
i+j≦6.

7. The anionic compounds as claimed in claim 1, wherein the radical —R₁— before attachment to the radical [Q] or to the radical [AA] is —CH₂—COOH.

8. The anionic compounds as claimed in claim 1, wherein the radical —R′₁ is a radical —CH₂COOH.

9. The anionic compounds as claimed in claim 1, wherein the amino acids are chosen from alpha-amino acids.

10. The anionic compounds as claimed in claim 9, wherein the alpha-amino acids are chosen from natural alpha-amino acids.

11. The anionic compounds as claimed in claim 10, wherein the natural alpha-amino acids are chosen from hydrophobic amino acids chosen from the group comprising tryptophan, leucine, alanine, isoleucine, glycine, phenylalanine, tyrosine and valine, in their L, D or racemic forms.

12. The anionic compounds as claimed in claim 11, wherein the natural alpha-amino acids are chosen from polar amino acids chosen from the group comprising aspartic acid, glutamic acid, lysine and serine, in their L, D or racemic forms.

13. The anionic compounds as claimed in claim 1, wherein the radical —R₂ is derived from a hydrophobic alcohol.

14. The anionic compounds as claimed in claim 1, wherein the radical —R₂ is derived from a hydrophobic acid.

15. The anionic compounds as claimed in claim 1, wherein at least one saccharide unit is in cyclic form.

16. The anionic compounds as claimed in claim 1, wherein at least one saccharide unit is in open reduced or open oxidized form.

17. The anionic compounds as claimed in claim 1, wherein at least one saccharide unit is chosen from the group of hexoses.

18. The anionic compounds as claimed in claim 1, wherein the backbone is made up of a discrete number of between 3 and 5 saccharide units.

19. The anionic compounds as claimed in claim 1, wherein the backbone is made up of a discrete number u=3 saccharide units.

20. The anionic compounds as claimed in claim 1, wherein the backbones are obtained by enzymatic degradation of a polysaccharide followed by purification.

21. The anionic compounds as claimed in claim 1, wherein the backbones are obtained by chemical degradation of a polysaccharide followed by purification.

22. The anionic compounds as claimed in claim 1, wherein the backbones are obtained chemically, by covalent coupling of lower-molecular-weight precursors.

23. A pharmaceutical composition which comprises an anionic compound as claimed in claim 1 and an active ingredient which is chosen from the group consisting of proteins, glycoproteins, peptides and nonpeptide therapeutic molecules.
